(19) United States
(12) Patent Application Publication
(10) Pub. No.: US 2010/0016247 A1
(43) Pub. Date: Jan. 21, 2010

(54) USING BUDDING YEAST TO SCREEN FOR INHIBITORS OF AURORA KINASES

(75) Inventors: YANCHANG WANG, Tallahassee, FL (US); SHU-YI SI, Beijing (CN)

Correspondence Address: Vedder Price, PC, 875 15th Street, NW, Suite 725, Washington, DC, 20005, US

(73) Assignee: FLORIDA STATE UNIVERSITY RESEARCH FOUNDATION, Tallahassee, FL (US)

(21) Appl. No.: 12/464,367

(22) Filed: May 12, 2009

Related U.S. Application Data
(60) Provisional application No. 61/082,238, filed on Jul. 21, 2008.

(51) Int. Cl. A61K 31/706; C12Q 1/18; G01N 33/569; C12Q 1/48

(52) U.S. Cl. 514/27; 435/32; 435/7.31; 435/15

(57) ABSTRACT

Methods of screening for novel Aurora kinase inhibitors in higher organisms are provided using hypomorphic ipl1 mutant yeast cells. Putative Aurora kinase inhibitors identified by present screening methods may be useful in treating individuals having a proliferative disease, such as cancer. Chemical compounds identified by present methods as selectively inhibiting growth of hypomorphic ipl1 mutant yeast cells may be used in compositions. Compositions or compounds identified by such screening methods may be administered to an individual in need thereof.

### CROSS-REFERENCE TO RELATED APPLICATIONS

This application claims the priority of U.S. Provisional Patent Application No. 61/082,238 entitled, “USING BUDDING YEAST TO SCREEN FOR INHIBITORS OF AURORA KINASES,” filed Jul. 21, 2008, the entire contents and disclosure of which are hereby incorporated by reference.

### GOVERNMENT INTEREST STATEMENT

This invention was made with support from the State of Florida under Florida Department of Health Grant No. 04NIR13. This invention was also made with support from the International Cooperation Program of the National Natural Science Foundation of China (Grant No. 30440420592). The State of Florida and the People's Republic of China may have rights to this invention. This invention was also made with support from the American Cancer Society (ACS) Research Scholar Grant No. RSG-08-104-010CCG.

### FIELD OF THE INVENTION

The present invention broadly relates to methods of screening for compounds that inhibit an Aurora kinase enzyme using a homologous and highly conserved protein from yeast, Ipl1. The present invention also broadly relates to compounds identified by such screens including, for example, Jadomycin B. In addition, the present invention further broadly relates to methods for administering compounds identified by such screens to animals or humans to treat, manage, inhibit, prevent, etc., a proliferative disease.

### BACKGROUND

Chemotherapeutic agents that target microtubules have been used for many years. These include the taxanes (e.g. paclitaxel, taxol, etc.), the epothilones, and the discodermolides, which stabilize microtubulues, and the Vinca alkaloids (vincristine, vinblastine, and vindestine), nocodazole, and colchicines, all of which destabilize microtubules. While these drugs may be effective in many circumstances, they may not discriminate well between dividing and nondividing cells and may display various side effects and complications. See, e.g., Andrews, P., “Aurora Kinases: shining lights on the therapeutic horizon?,” Oncogene 24:5005-5015 (2005), the entire content and disclosure of which is hereby incorporated by reference. A need continues in the art for new compounds and therapies that selectively target abnormally dividing cells.

### SUMMARY

According to a first broad aspect of the present invention, a method is provided comprising the following steps: (a) measuring an amount of growth over a predetermined period of time of a population of wild-type yeast cells in or on a first medium containing a test compound and an amount of growth over the predetermined period of time of a population of hypomorphic ipl1 mutant yeast cells in or on a second medium containing the test compound; and (b) comparing the amount of growth of the population of hypomorphic ipl1 mutant yeast cells to the amount of growth of the population of wild-type yeast cells to thereby determine whether the test compound is a putative Aurora kinase inhibitor.

According to a second broad aspect of the present invention, a method is provided comprising the following steps: (a) measuring over a predetermined period of time: (i) an amount of growth of a first population of wild-type yeast cells in or on a first medium containing a test compound; (ii) an amount of growth of a first population of hypomorphic ipl1 mutant yeast cells in or on a second medium containing the test compound; (iii) an amount of growth of a second population of wild-type yeast cells in or on a third medium lacking the test compound; and (iv) an amount of growth of a second population of hypomorphic ipl1 mutant yeast cells in or on a fourth medium lacking the test compound; (b) calculating a growth differential for wild-type yeast cells by calculating the difference between the amount of growth of the first population of wild-type yeast cells and the amount of growth of the second population of wild-type yeast cells and a growth differential for hypomorphic ipl1 mutant yeast cells by calculating the difference between the amount of growth of the first population of hypomorphic ipl1 mutant yeast cells and the amount of growth of the second population of hypomorphic ipl1 mutant yeast cells; and (c) comparing the growth differential for the population of hypomorphic ipl1 mutant yeast cells to the growth differential for the population of wild-type yeast cells to thereby determine whether the test compound is a putative Aurora kinase inhibitor.

According to a third broad aspect of the present invention, a method is provided comprising the following steps: (a) measuring an amount of growth over a predetermined period of time of a first population of hypomorphic ipl1 mutant yeast cells in or on a first medium containing a test compound and an amount of growth over the predetermined period of time of a second population of hypomorphic ipl1 mutant yeast cells in or on a second medium lacking the test compound; and (b) comparing the amount of growth of the first population of hypomorphic ipl1 mutant yeast cells to the amount of growth of the second population of hypomorphic ipl1 mutant yeast cells to thereby determine whether the test compound is a putative Aurora kinase inhibitor.

According to a fourth broad aspect of the present invention, a pharmaceutical composition is provided comprising a therapeutically effective amount of a test compound in combination with a pharmaceutically acceptable carrier, wherein the test compound has been determined to be a putative Aurora kinase inhibitor according to present screening methods. According to some embodiments, the test compound may be Jadomycin B.

According to a fifth broad aspect of the present invention, a method is provided comprising the following steps: (a) identifying an individual experiencing a form of cancer; and (b) administering to the individual a composition comprising a therapeutically effective amount of a test compound, wherein the test compound has been determined to be a putative Aurora kinase inhibitor according to present screening methods.

According to a sixth broad aspect of the present invention, a method is provided comprising the following steps: (a) identifying an individual in need of modulation of an Aurora kinase enzyme; and (b) administering to the individual a composition comprising a therapeutically effective amount of a test compound, wherein the test compound has been determined to be a putative Aurora kinase inhibitor according to present screening methods.

### BRIEF DESCRIPTION OF THE DRAWINGS

The invention will be described in conjunction with the accompanying drawings, in which:

FIG. 1A is an image showing the structure of a subunit of Aurora-B co-crystallized with Hesperadin rendered as a ball-and-stick model where the structure is based on Protein Database Bank entry 2bfy as used for the structure-based virtual screening;

FIG. 1B is an image (generated by SYBYL7.1) showing the predicted docking model of Jadomycin B to the ATP-binding pocket of Aurora-B kinase, where Jadomycin B is rendered as a stick model, with the molecular surface of the kinase domain being shown with electrostatic potentials;

FIG. 1C is an image (generated by SYBYL7.1) showing the specific hydrogen bonds (broken lines) formed between Jadomycin B (stick model) and residues (ball-and-stick model) around the ATP-binding site or pocket:

FIG. 1D shows the chemical structure of Jadomycin B;

FIG. 2A is an amino acid sequence listing of Ipl1 and Aurora-B kinase showing the ATP-binding pocket of Aurora kinase is well conserved with the shadows representing the residues surrounding the ATP-binding site and panes representing the different residues between Ipl1 and Aurora-B kinase;

FIG. 2B is a set of bar graphs showing the relative percentages of growth measured as OD₆₀₀ for wild-type and ipl1-321 yeast cells treated with 10 μg/ml Jadomycin B and other compounds at 25° C. for 24 hours, versus untreated cells with the results presented as means and standard deviations from three separate experiments (p<0.05);

FIG. 2C is a set of bar graphs showing the relative percentage of growth measured as OD₆₀₀ as described in FIG. 2B of wild-type and ipl1-321 cells treated with Jadomycin B at final concentrations of 0.4, 2, 5, 20, and 100 μg/ml of Jadomycin B after incubation at 25° C. for 24 hours;

FIG. 3A is a graphical plot showing the inhibition of Aurora-B kinase with various concentrations of Jadomycin B in the presence of 25 μM ATP;

FIG. 3B is a graphical plot showing ATP-dependent IC₅₀ values for inhibition of Aurora B kinase by Jadomycin B in the presence of various concentrations of ATP;

FIG. 4A are bar graphs showing IC₅₀ values for A549, Hela, and MCF-7 cells exposed to increasing concentrations of Jadomycin B, Jadomycin S, and Jadomycin T for 48 hours with values determined as described herein in mean±SD from three independent experiments;

FIG. 4B are graphical plots showing the number of A549, Hela, and MCF-7 cells counted every 24 hours as described herein after treatment with or without different concentrations of Jadomycin B (JB) for 96 hours and detachment by trypsinization;

FIG. 5A are images showing results of FACS analysis as described herein for A549 cells treated with 0 or 5 μg/ml Jadomycin B and collected and fixed at indicated times with dotted line representing portion of apoptotic cells and percentage of apoptotic cells plotted over 48 hours;

FIG. 5B is an image and corresponding graphical plots showing the results of FACS analysis of A549 cells treated with 0 or 5 μg/ml Jadomycin B and harvested at the indicated times with the percentages of viable population of treated and untreated cells in each phase of the cell cycle calculated and presented in mean±SD from three independent experiments;

FIG. 5C is an image showing the apoptotic bodies of cells stained with Hoechst 33342 and examined by fluorescence microscopy;

FIG. 6A is an image of Western blots probed with anti-phospho-H3 (Ser 10) antibody and control anti-β-tubulin antibody using 30 μg of protein from each sample derived from A459 cell cultures harvested after 24 hours in the absence or presence of different concentrations of Jadomycin B;

FIG. 6B is an image of Western blots probed with anti-phospho-H3 (Ser 10) antibody and control anti-β-tubulin antibody using 30 μg of protein from each sample derived from Hela and HepG2 cell cultures harvested after 24 hours in the absence or presence of 10 μg/ml of Jadomycin B; and

FIG. 6C is a bar graph showing the quantitative analysis of the inhibition of H3 phosphorylation by Jadomycin B in A549 cells measured by grayscale scanning after Western blotting as a percentage ratio of H3 phosphorylation in the presence of different concentrations of Jadomycin B, relative to untreated cells, with data presented in mean±SD (n=3).

### DETAILED DESCRIPTION OF THE PREFERRED EMBODIMENTS

### Definitions

It is advantageous to define several terms before describing the invention. It should be appreciated that the following definitions are used throughout this application.

For the purposes of the present invention, the term “comprising” means various compositions, compounds, molecules, components, capabilities and/or steps, etc., may be conjointly employed in embodiments of the present invention. Accordingly, the term “comprising” encompasses the more restrictive terms “consisting essentially of” and “consisting of.”

For the purposes of the present invention, the term “compound” as used herein encompasses all types of organic or inorganic molecules, including but not limited to proteins, peptides, polysaccharides, lipids, nucleic acids, small organic molecules, inorganic compounds, and derivatives thereof.

For the purposes of the present invention, the term “test compound” refers to a compound that is to be tested by screening methods according to embodiments of the present invention.

For the purposes of the present invention, the terms “patient,” “subject,” or “individual” as used herein may be used interchangeably and generally refer to any animal, preferably mammals, which may be subject to treatment with compounds identified by screening methods according to embodiments of the present invention. The terms “patient,” “subject,” or “individual” may refer to any animal or human suffering from a proliferative disease, such as cancer. Alternatively, the terms “patient,” “subject,” or “individual” may also refer to any animal or human having abnormally growing or dividing cells or any animal or human in need of modulation of an Aurora kinase enzyme.

For the purposes of the present invention, the terms “mammal,” “mammals,” and “mammalian” are intended to include any animal classified phylogenetically or generally understood to be a mammal. For example, the terms “mammal,” “mammals,” and “mammalian” may refer to humans, monkeys, and other primates. The terms “mammal” and “mammalian” may also refer to animals having an agricultural or domesticated use or purpose including, but not limited to, cattle, sheep, goats, pigs, horses, canines, cats, etc., as well as animals having research or laboratory uses including, but not limited to, rabbits, mice, rats, etc. The terms “mammal” and “mammalian” may further include any wild-type, mutated, or transgenic mammal, as well as any genetic lines of mammals. In general, the term “mammalian cells” may be any cells present in a mammal or derived from a mammal. The terms “mammalian cell line,” “mammalian cell culture,” or “culture of mammalian cells” refer interchangeably to cells in a culture which are understood to be a mammalian cell line or a mammalian cell culture by one of skill in the art or any cells in a culture that were originally derived from a mammal.

For the purposes of the present invention, the term “proliferative disease” refers to any disease caused by unregulated growth of cells causing symptoms in an individual, subject, or patient. For example, the term “proliferative disease” may refer to any form of cancer.

For the purposes of the present invention, the terms “Aurora kinase” or “Aurora kinases” refer to any kinase gene or protein known or understood in the art to be an Aurora kinase gene or protein. Aurora kinases include kinase genes or proteins within the Aurora-A, Aurora-B, and Aurora-C families of kinases. “Aurora kinase” or “Aurora kinases” may include any Aurora kinase gene or protein, or homologue thereof, present in eukaryotic cells, such as mammalian cells. “Aurora kinase” or “Aurora kinases” may also refer to any wild-type, truncated, mutated, tagged, fusion, molecularly altered, etc., version of an Aurora kinase gene or protein. Aurora kinases may also include other Serine/Threonine kinases, such as newly discovered Serine/Threonine kinases, that function during mitosis and have a sufficiently high degree of homology to any known Aurora kinase gene or protein.

For the purposes of the present invention, the term “inhibitor” refers to any composition, compound, etc., that inhibits the activity and/or expression level, substrate binding, etc., of an enzyme in vivo, in vitro, and/or in a cell or tissue culture according to known assays.

For purposes of the present invention, the term “Aurora kinase inhibitor” refers to any composition, compound, etc., that inhibits the activity, expression, and/or level of an Aurora kinase in vivo, in vitro, and/or in a cell or tissue culture according to known assays.

For purposes of the present invention, the term “putative Aurora kinase inhibitor” refers to a composition, compound, etc., that is identified by embodiments of the present screening methods to inhibit growth of ipl1 mutant yeast cells. For example, the term “putative Aurora kinase inhibitor” may refer to a composition, compound, etc., that is identified by embodiments of the present screening methods to selectively inhibit growth of ipl1 mutant yeast cells relative to wild-type yeast cells.

For the purposes of the present invention, the terms “ATP-binding site” or “ATP-binding pocket” refer interchangeably to the portion of an Aurora kinase enzyme or protein, or fragment thereof, that binds or accommodates an ATP molecule for hydrolysis typically to provide energy for a reaction and/or to provide phosphates for phosphorylation of a kinase target, such as an Aurora kinase target.

For the purposes of the present invention, the term “population” in reference to cells generally refers to a clonal group of cells. However, the term “population” may also include a single cell that is capable of dividing under appropriate growth conditions to form a clonal group of cells. Under some conditions, a “population” of cells may include an absence of cells if the conditions, including any exposure to a test compound, negatively affect the viability, senescence, programmed cell death, etc. of the cells.

For the purposes of the present invention, the terms “hypomorphic,” “hypomorphic allele,” or “hypomorphic mutation” refer interchangeably to a mutant allele or version of a gene that results in a partial loss of function of such gene relative to wild-type as generally understood by those skilled in the art. For example, a “hypomorphic allele” or “hypomorphic mutation” may refer to a version of a gene having a lower expression level and/or level of activity of its gene product relative to wild-type levels. A “hypomorphic allele” or “hypomorphic mutation” may include temperature-sensitive alleles or mutations for a gene. The term “hypomorphic” or “hypomorph” may also refer to a whole organism having a hypomorphic mutation. The terms “hypomorphic allele” or “hypomorphic mutation” are to be distinguished from “null” mutations or alleles. A null mutation or allele refers to a complete (or nearly complete) loss of function of such gene.

For the purposes of the present invention, the terms “wild-type yeast cell” or “wild-type yeast cells” refer to any yeast cell(s) having at least one wild-type allele of the IPL1 gene. The terms “wild-type yeast cell” or “wild-type yeast cells” may also refer to any yeast cell(s) containing one or more mutations, such as silent mutations, in either the regulatory or coding regions of an IPL1 gene as long as the activity, level, or expression of the Ipl1 product of such gene is not significantly altered or modified.

For the purposes of the present invention, the terms “culture” or “cell culture” generally refer to a medium containing a population of cells.

For the purposes of the present invention, the term “medium” in reference to the growth of cells generally refers to a liquid sample, solution, suspension, etc., or on a solid substrate containing nutrients essential for growth of a population of cells. An example of a liquid medium may be a YPD medium, whereas an example of a solid medium may include agar plates or dishes.

For the purposes of the present invention, the terms “containing,” “contain,” or “contained” in reference to a culture, solution, medium, etc., refer to compounds, substances, cells, etc., that are placed in or on the culture, solution, medium, etc.

For the purposes of the present invention, the term “growth” generally refers to an increase in the number and/or size of cells in a population or culture of cells. The term “growth” may encompass any or all factors affecting the number and/or size of cells in a population or culture of cells, including their viability, rate of division, growth in cell size, senescence, programmed cell death, etc., in response to their environment or growth conditions including the presence of any test compound(s) or putative Aurora kinase inhibitor(s).

For the purposes of the present invention, the term “amount of growth” refers to any quantitative measurement of growth, as a function of time, of either treated or untreated yeast cells, such as wild-type or ipl1 mutant yeast cells, or mammalian cells in culture. For example, such an “amount of growth” may be measured by an increase in the number or area of cells on a solid medium or by an increase in the number or density of cells in a liquid medium. The term “growth” may be measured in terms of total change in the number and/or size of cells in a population or culture of cells over a period of time or in terms of rate of change in the number and/or size of cells in a population or culture of cells during a period of time. The term “amount of growth” may also refer to a qualitative determination of growth, such as by sight, where differences of growth between different populations of cells are obvious by inspection.

For the purposes of the present invention, the term “material” in reference to the application of a test compound to the surface of a solid medium refers to an absorbent material that may be used to absorb a solution containing the test compound and overlaid onto the surface of a solid medium as a way for applying the test compound to at least an area of the solid medium. For example, the “material” may be a form of paper. The “material” may be either applied to the whole surface of a solid medium or only to a portion of the solid medium.

For the purposes of the present invention, the term “zone of inhibition” refers to an area or zone of inhibited growth of cells at, near, or surrounding a point or position where a test compound is applied to a solid medium, such as by overlaying a piece of material soaked in a solution containing the test compound onto the surface of the solid medium. Since the concentration of test compound may decrease at greater distances from the point or position where the test compound is applied to the solid medium, test compounds that are more potent at inhibiting growth of yeast cultures may produce a relatively larger area or zone of inhibition (i.e., it will inhibit growth at lower concentrations), whereas test compounds that are less potent at inhibiting growth of yeast cultures may produce a relatively smaller area or zone of inhibition (i.e., it will inhibit growth at only higher concentrations). A zone of inhibition may be measured in terms of an area, diameter, radius, circumference, etc., or any combination thereof. A zone of inhibition may be defined as an area, etc., of total inhibition of growth (i.e., no yeast cells are present), or an area, etc., of partial inhibition having a reduced density, number, and/or sizes of cells below a certain amount or threshold.

For the purposes of the present invention, the term “growth differential” refers to the difference in the amounts of growth of treated and untreated cells of the same type. For example, the “growth differential” for wild-type yeast cells and hypomorphic ipl1 mutant yeast cells, such as ipl1-321 or ipl1-2 mutant yeast cells, may be defined as the difference in growth between treated and untreated populations of wild-type yeast cells and populations of hypomorphic ipl1 mutant yeast cells, respectively. Such a “growth differential” may be compared as an absolute value of the difference in the amounts of growth of treated and untreated cells of the same type.

For the purposes of the present invention, the term “absolute value” refers to a numerical value of a measurement, amount, calculation, etc., without regard to its sign (i.e., without regard to whether the numerical value is a positive or negative number). Such numerical value of a measurement, amount, calculation, etc., may be any real number. For example, the number 5 is the absolute value of both 5 and −5.

For the purposes of the present invention, the term “permissive temperature” refers to a temperature or range of temperatures at which the growth of mutant yeast cells is about the same as wild-type yeast cells under normal conditions. For example, the “permissive temperature” may be a temperature within a range of about 23° C. to about 26° C. For example, the “permissive temperature” may be about 25° C.

For the purposes of the present invention, the term “restrictive temperature” refers to a temperature or range of temperatures at which the growth of mutant yeast cells is less than the growth of wild-type yeast cells under normal conditions or a temperature or range of temperatures at which mutant yeast cells do not grow under normal conditions. For example, the “restrictive temperature” may be a temperature above about 29° C., or alternatively, the “restrictive temperature” may be a temperature above about 35° C. For example, the “restrictive temperature” may be about 37° C.

For the purposes of the present invention, the term “partially restrictive temperature” refers to a temperature or range of temperatures at which the growth of mutant yeast cells is about the same as wild-type yeast cells under normal conditions. However, the term “partially restrictive temperature” may further refer to a temperature or range of temperatures at which the growth of mutant yeast cells is more sensitive to additional factors, compounds, etc., such as a test compound, which may disproportionately affect the growth of the mutant yeast cells relative to the growth of wild-type yeast cells. For example, the “partially restrictive temperature” may be a temperature within a range of about 26° C. to about 35° C., or alternatively, within a range of about 26° C. to about 29° C.

For the purposes of the present invention, the term “normal conditions” for yeast cells refers to conditions for the growth of yeast cells in or on a medium in the absence of compounds, factors, etc., such as a test compound, that may negatively affect the growth of yeast cells.

For the purposes of the present invention, the terms “apoptotic cell,” “apoptotic cells,” apoptotic body,” or “apoptotic bodies” refer interchangeably to features or characteristics of cells or remnants of cells that have undergone or are undergoing apoptosis. Apoptotic cells may be characterized by a variety of morphological changes, including, for example, membrane blebbing, loss of membrane asymmetry and attachment, cell shrinkage, nuclear fragmentation, chromatin condensation, chromosomal DNA fragmentation, etc. Such features or characteristics are well known in the art and may be determined, for example, by microscopy to inspect the appearance of cells or by FACS analysis to determine if cells or remnants of cells contain sub-G1 phase or sub-2N DNA content.

For the purposes of the present invention, the terms “product of a gene” or “gene product” in reference to a particular gene may refer interchangeably to any RNA or polypeptide molecule encoded by such gene.

For the purposes of the present invention, the term “optical density” or “OD” refers to the fraction of incident light absorbed by a sample, solution, or suspension per a given distance or thickness of the sample, solution, or suspension. For example, OD₆₀₀ refers to the absorption of light having a wavelength of 600 nm.

For the purposes of the present invention, the term “carrier” generally refers to inert substances that may be formulated with a drug, test compound, putative Aurora kinase inhibitor, etc. for pharmaceutical applications. The term “carrier” may also include a “delivery reagent” as defined herein.

For the purposes of the present invention, the term “therapeutically effective amount” refers to an amount of a compound effective to achieve a desired result or purpose or therapeutic benefit, including the effective treatment, inhibition, prevention, management, etc., of a proliferative disease, such as cancer. The term “therapeutically effective amount” may refer to any amount effective to prevent, alleviate, abate, or otherwise reduce the severity of symptoms in an individual, subjects or patient, such as an amount that (i) selectively or preferentially kills diseased or cancerous cells or inhibits their growth in the body of an individual, subject, or patient, such as by causing such diseased or cancerous cells to undergo apoptosis, etc., or (ii) selectively or preferentially causes diseased or cancerous cells in the body of an individual, subject, or patient to enter a state of quiescence, to stop dividing or enter cell cycle arrest, or to lose their malignant characteristics. For example, a “therapeutically effective amount” may refer to any amount necessary or sufficient to measurably inhibit the activity and/or expression of an Aurora kinase enzyme, such as an Aurora kinase enzyme in a diseased or cancerous cell or tissue in the body of an individual, subject, or patient. A “therapeutically effective amount” may also refer to any amount necessary or sufficient to cause cell cycle or growth arrest or to cause apoptosis of a diseased or cancerous cell in the body of an individual, subject, or patient. The term “therapeutically effective amount” may refer to a single dose taken alone or as part of a dosage regimen comprising multiple doses administered over a period of time. Factors to consider when determining a “therapeutically effective amount” or “effective amount” for treatment with a compound may include the manner/route of administration, timing of administration, rate of excretion, target site, disease or physiological state, medical history, age, sex, physical characteristics, other medications, etc. This list of factors is illustrative and not exhaustive, and may include any or all factors which might be considered by a skilled scientist, veterinarian, or physician (as the case may be) in determining an appropriate treatment.

### Description

In mammals, there are three Aurora kinases, Aurora-A, -B, and -C, which function as serine/threonine kinases crucial for cell cycle control. See, e.g., Fu, J., “Roles of Aurora Kinases in Mitosis and Tumorigenesis,” Mol Cancer Res 5(1):1-10 (2007), the entire content and disclosure of which is hereby incorporated by reference. Although the catalytic domains of these three kinases are highly conserved, they show distinct subcellular localization and biological function. See, e.g., Kimmins S et al., “Differential functions of the Aurora-B and Aurora-C kinases in mammalian spermatogenesis,” Mol Endocrinol 21:726-39 (2007); Meraldi P et al., “Aurora kinases link chromosome segregation and cell division to cancer susceptibility,” Curr Opin Genet Dev 14:29-36 (2004); Andrews, P, “Aurora kinases: shining lights on the therapeutic horizon?,” 24:5005-5015 (2005), the entire contents and disclosures of which are hereby incorporated by reference.

Aurora-A, which is required for centrosome maturation and separation, localizes to centrosomes from early S phase to late M phase. See, e.g., Schumacher J M et al. “AIR-2: An Aurora/Ipl1-related protein kinase associated with chromosomes and midbody microtubules is required for polar body extrusion and cytokinesis in Caenorhabditis elegans embryos,” J Cell Biol 143:1635-46 (1998), the entire content and disclosure of which is hereby incorporated by reference. Aurora-B is a component of chromosomal passenger complex (CPC), whose function in mitosis has been extensively studied. In addition to Aurora-B, the CPC contains Survivin, Borealin and inner centromere protein (INCENP). See, e.g., Gassmann R et al., “Borealin: a novel chromosomal passenger required for stability of the bipolar mitotic spindle,” J Cell Biol 166:179-91 (2004); Skoufias D A et al., “Human survivin is a kinetochore-associated passenger protein,” J Cell Biol 151:1575-82 (2000); Adams R R et al., “Chromosomal passengers and the (aurora) ABCs of mitosis,” Trends Cell Biol 11:49-54 (2001), the entire contents and disclosures of which are hereby incorporated by reference. The CPC associates with centromeric regions of chromosomes in the early stages of mitosis, but it translocates to microtubules after the onset of anaphase. When cells undergo cytokinesis, the CPC accumulates at the spindle midzone and finally concentrates at the midbody. As a serine/threonine kinase, Aurora-B phosphorylates a number of targets including, for example, histone H3 at Ser10, although the functional significance of this modification remains uncertain. See, e.g., Hsu J Y et al., “Mitotic phosphorylation of histone H3 is governed by Ipl1/aurora kinase and Glc7/PP1 phosphatase in budding yeast and nematodes,” Cell 102:279-91 (2000), the entire content and disclosure of which is hereby incorporated by reference. Recent data from both yeast and mammals suggest that Aurora-B kinase activity may be necessary for correct microtubule-kinetochore attachments, chromosome alignment and segregation, as well as cytokinesis. See, e.g. Ruchaud S et al., “Chromosomal passengers: conducting cell division,” Nat Rev Mol Cell Biol 8:798-812 (2007), the entire content and disclosure of which is hereby incorporated by reference.

Aurora-C is highly expressed in testis along with the other two human Aurora kinases and may play a role in tumorigenesis, especially in the absence of p53. See, e.g., Dutertre S et al., “The absence of p53 aggravates polyploidy and centrosome number abnormality induced by Aurora-C overexpression,” Cell Cycle 4:1783-7 (2005); Tang C J et al., “Dynamic localization and functional implications of Aurora-C kinase during male mouse meiosis,” Dev Biol 290:398-410 (2006), the entire contents and disclosures of which are hereby incorporated by reference. Recent data indicate that Aurora-C acts like Aurora-B in its localization during mitosis, and it is able to complement Aurora-B kinase function. See, e.g. Sasai K et al., “Aurora-C kinase is a novel chromosomal passenger protein that can complement Aurora-B kinase function in mitotic cells,” Cell Motil Cytoskeleton 59:249-63 (2004), the entire content and disclosure of which is hereby incorporated by reference.

Accumulating evidence indicates that Aurora kinases may be over-expressed in a wide range of tumor cells, including breast cancer (see, e.g., Sen, S. et al., “A putative serine/threonine kinase encoding gene BTAK on chromosome 20q13 is amplified and overexpressed in human breast cancer cell lines,” Oncogene 14:2195-2200 (1997); Miyoshi Y et al., “Association of centrosomal kinase STK15/BTAK mRNA expression with chromosomal instability in human breast cancers,” Int J Cancer 92:370-3 (2001), the entire contents and disclosures of which are hereby incorporated by reference), colon cancer (see, e.g. Bischoff, J. R. et al., “A homologue of Drosophila aurora kinase is oncogenic and amplified in human colorectal cancers,” EMBO J 17:3052-65 (1998); Katayama, H. et al., “Mitotic kinase expression and colorectal cancer progression,” J Natl Cancer Inst 91:1160-2 (1999); Takahashi. T. et al., “Centrosomal kinases, HsAIRK1 and HsAIRK3, are overexpressed in primary colorectal cancers,” Jpn J Cancer Res 91:1007-14 (2000), the entire contents and disclosures of which are hereby incorporated by reference), pancreatic cancer (see, e.g. Li, D. et al., “Overexpression of oncogenic STK15/BTAK/Aurora A kinase in human pancreatic cancer” Clin Cancer Res 9:991-7 (2003), the entire content and disclosure of which is hereby incorporated by reference), ovarian cancer (see, e.g., Gritsko, T. M. et al., “Activation and overexpression of centrosome kinase BTAK/Aurora-A in human ovarian cancer,” Clin Cancer Res 9:1420-6 (2003), the entire content and disclosure of which is hereby incorporated by reference), and gastric cancer (see, e.g., Sakakura, C. et al., “Tumour-amplified kinase BTAK is amplified and overexpressed in gastric cancers with possible involvement in aneuploid formation. Br J Cancer 84:824-31 (2001), the entire content and disclosure of which is hereby incorporated by reference). See also, e.g. Wang, Y., “Chromosome instability in yeast and its implications to the study of human cancer,” Front Biosci 13:2091-102 (2008); Malumbres, M. et al., “Cell cycle kinases in cancer,” Curr Opin Genet Dev 17:60-5 (2007), the entire contents and disclosures of which are hereby incorporated by reference.

A recent systematic analysis of Aurora kinase mRNA levels in multiple primary tumors may indicate that Aurora-A and -B are significantly over-expressed. See, e.g., Lin, Y. S. et al., “Gene expression profiles of the aurora family kinases,” Gene Expr 13:15-26 (2006), the entire content and disclosure of which is hereby incorporated by reference. Since aberrant Aurora kinases may lead to errors in chromosome alignment and segregation, Aurora kinases may be perceived as promising targets for antitumor drugs. Moreover, Aurora kinases are generally only expressed during mitosis. Thus, inhibition of Aurora kinases may have relatively little effect on quiescent cells making Aurora kinases very attractive targets for anticancer therapy. See, e.g., Walter, A. O. et al., “The mitotic serine/threonine kinase Aurora2/AIK is regulated by phosphorylation and degradation,” Oncogene 19:4906-16 (2000), the entire content and disclosure of which is hereby incorporated by reference. Aurora kinases have emerged as promising targets for cancer therapy because of their critical role in mitosis. It is thought that inhibitors of Aurora kinases may have enormous potential for the treatment of cancer since Aurora kinases are expressed and active in dividing cells. However, to discover novel inhibitor compounds of Aurora kinases, new approaches that are capable of high-throughput analysis must be undertaken.

One effective approach to inhibit kinases may be to block their interaction with substrates. Molecules that show similar structure to kinase substrates may function as competitive inhibitors. For example, three small-molecular inhibitors of Aurora kinases, ZM447439, Hesperadin, and VX-680, have recently been described. Gadea et al., “Aurora kinase inhibitor ZM447439 blocks chromosome-induced spindle assembly, the completion of chromosome condensation, and the establishment of the spindle integrity checkpoint in Xenopus egg extracts,” Mol Biol Cell 16:1305-18 (2005); Hauf, S. et al., “The small molecule Hesperadin reveals a role for Aurora B in correcting kinetochore-microtubule attachment and in maintaining the spindle assembly checkpoint,” J Cell Biol 161:281-94 (2003); Harrington, E. A. et al., “VX-680, a potent and selective small-molecule inhibitor of the Aurora kinases, suppresses tumor growth in vivo,” Nat Med 10:262-7 (2004), the entire contents and disclosures of which are hereby incorporated by reference. All three molecules may show antitumor activity in vitro and some in vivo, indicating the great potential of Aurora kinase inhibitors as antitumor drugs. See also, e.g., Ditchfield, C. et al., “Aurora B couples chromosome alignment with anaphase by targeting BubR1, Mad2, and Cenp-E to kinetochores,” J Cell Biol 161:267-80 (2003); Morrow, C. J. et al., “Bub1 and aurora B cooperate to maintain BubRI-mediated inhibition of APC/Cdc20,” J Cell Sci 118:3639-52 (2005), the entire contents and disclosures of which are hereby incorporated by reference. However, each Aurora kinase inhibitor identified may show varying degrees of inhibition and target specificity as well as varying effects on the cell cycle and apoptosis. Therefore, a need continues in the art for the identification of novel compounds that inhibit Aurora kinases to allow for greater selection of compounds having the highest degree of target specificity and effectiveness with the fewest side effects.

Aurora kinases are well conserved in all eukaryotes. See, e.g. Brown, J. R. et al., “Evolutionary relationships of Aurora kinases: implications for model organism studies and the development of anti-cancer drugs,” BMC Evol Biol 4:39 (2004); Kimura, M. et al., “Cell cycle-dependent Expression and Spindle Pole Localization of a Novel Human Protein Kinase, Aik, Related to Aurora of Drosophila and Yeast Ipl1,” J Biol Chem 272(21):13766-71 (1997), the entire contents and disclosures of which are hereby incorporated by reference. The IPL1 gene encodes the single Aurora kinase in budding yeast and is thought to function similarly to Aurora B at yeast centromeres. See, e.g., Tanaka, T. V. et al., “Evidence that the Ipl1-Sli15 (Aurora kinase-INCENP) complex promotes chromosome bi-orientation by altering kinetochore-spindle pole connections” Cell 108:317-29 (2002), the entire content and disclosure of which is hereby incorporated by reference.

Embodiments of screening methods of the present invention described herein are designed to employ the Aurora kinase homologue from Saccharomyces cerevisiae, Ipl1, as a basis for screening potential inhibitors of Aurora kinases in higher organisms. Given the high degree of sequence homology and conservation in function between Ipl1 and Aurora kinases, it is expected that inhibitors of Ipl1 in yeast identified by present screening methods may also inhibit Aurora kinases in higher organisms. More particularly, embodiments of the present invention provide screening methods using hypomorphic ipl1 mutant yeast cells to identify putative Aurora kinase inhibitors. For example, such hypomorphic mutation or allele of ipl1 used in embodiments of present screening methods may include ipl1-321 described herein and also in, for example, Biggins et al., “The conserved protein kinase Ipl1 regulates microtubule binding to kinaetochores in budding yeast,” Genes Dev. 13: 532-544 (1999), the entire content and disclosure of which is hereby incorporated by reference. Alternatively, such hypomorphic mutations or alleles of ipl1 used in embodiments of present screening methods may include ipl1-2 described in, for example, Chan et al., “Isolation and Characterization of Chromosome-Gain and Increase-in-Ploidy Mutants in Yeast,” Genetics 135: 677-691 (1993), the entire content and disclosure of which is hereby incorporated by reference. However, it is to be understood that any known or later generated hypomorphic mutation or allele of ipl1 may potentially be used according to embodiments of present screening methods as long as any such hypomorphic mutation or allele of ipl1 does not significantly impact growth of yeast cells having such hypomorphic mutation or allele under normal or permissive conditions in the absence of treatment with a test compound.

Because yeast cells having a hypomorphic mutation or allele of ipl1 may have weakened Ipl1 kinase activity, compounds that effectively inhibit Ipl1 may be especially potent at hindering growth, division, and/or viability of ipl1 hypomorphic mutant yeast cells, such as ipl1-321 or ipl1-2, relative to wild-type yeast cells. Therefore, according to embodiments of present screening methods, compounds that selectively inhibit growth, division, and/or viability of ipl1 mutant yeast cells relative to wild-type yeast cells may comprise inhibitors of the Ipl1 kinase itself Furthermore, given the high degree of sequence homology and conserved function between Ipl1 and Aurora kinases, any compound identified by embodiments of present screening methods as a potential inhibitor of Ipl1 kinase may also be an inhibitor of an Aurora kinase(s) in higher organisms, such as animals and humans. For example, the successful identification of a compound, Jadomycin B, using an embodiment of present screening methods is described herein, which is shown to effectively inhibit Aurora kinase activity in vitro and Aurora kinase function in cells of higher organisms, thus providing evidence for the effectiveness of embodiments of the present screening methods for identifying Aurora kinase inhibitors.

Due to the availability of powerful genetic and biochemical tools, their short doubling time, and the ability to easily monitor their growth in culture, yeast cells may provide distinct advantages for extensive screening and mechanistic studies of anticancer drugs. Embodiments of the present invention seek to adopt these advantages of yeast by screening for drugs or compounds that specifically target Ipl1, a yeast homologue of Aurora kinases. Generally speaking, to test a compound according to embodiments of the screening methods of the present invention, wild-type and hypomorphic ipl1 mutant yeast cells may be exposed to a putative Ipl1 inhibitor or test compound, and growth, division, and/or viability of cells may be monitored or measured over a period of time or after expiration of a period of time. Alternatively, to test a compound according to embodiments of the screening methods of the present invention, hypomorphic ipl1 mutant yeast cells may be treated or untreated with a putative Ipl1 inhibitor or test compound, and growth, division, and/or viability of cells may be monitored or measured over a period of time or after expiration of a period of time. Since Ipl1 is necessary for proper segregation of chromosomes and progression through mitosis, test compound(s) that effectively inhibit Ipl1 may preferentially limit growth, division, and/or viability of hypomorphic ipl1 mutant yeast cells or cultures relative to wild-type yeast cells or untreated hypomorphic ipl1 mutant yeast cells. Thus, embodiments of screening methods of the present invention may provide a simple yet powerful method that is suitable for high-throughput screening of potential Aurora kinase inhibitors.

Although Aurora-B kinase is believed to be the primary target for therapy (see, e.g. Giet, R. et al., “Drosophila aurora B kinase is required for histone H3 phosphorylation and condensin recruitment during chromosome condensation and to organize the central spindle during cytokinesis,” J Cell Biol 152:669-82 (2001); Tanaka, T. V. et al., “Evidence that the Ipl1-Sli15 (Aurora kinase-INCENP) complex promotes chromosome bi-orientation by altering kinetochore-spindle pole connections,” Cell 108:317-29 (2002), the entire contents and disclosures of which are hereby incorporated by reference), embodiments of present screening methods may potentially be used to identify inhibitors of any of the three types or families of Aurora kinase enzymes (i.e., Aurora-A, -B, and -C). Inhibition of any of these Aurora kinases (or inhibition of any combination of these Aurora kinases) may prove to be an effective treatment or therapy for disease in any given individual, subject or patient. Given the high degree of similarity between Aurora kinases and Ipl1, inhibitors of Ipl1 identified by embodiments of the present screening methods may also inhibit any one or any combination of the three known Aurora kinase families in higher organisms.

According to embodiments of the screening methods of the present invention, a compound of interest or test compound may be introduced to cultures of both wild-type and hypomorphic ipl1 mutant yeast cells. For example, such hypomorphic ipl1 mutant yeast cells may include ipl1-321 or ipl1-2. Typically, wild-type and hypomorphic ipl1 mutant yeast cells are initially present at approximately equal amounts or concentrations in their respective mediums. After a predetermined period of time has elapsed, the amount of growth and/or number of cells within each culture may be determined. According to embodiments of present screening methods, yeast cells may be grown at a permissive temperature, such as any temperature in a range of about 23° C. to about 26° C., or at any temperature where the rate of growth of wild-type and hypomorphic ipl1 mutant yeast cells is the same or approximately the same in the absence of a test compound. For example, such permissive temperature may be about 25° C. The predetermined period of time to allow for growth of yeast cells may vary depending on conditions and the volume of medium used. For example, a predetermined period of time of about 24 hours may be used.

According to other embodiments of present screening methods, a compound of interest or test compound may be introduced to a culture of hypomorphic ipl1 mutant yeast cells, and the amount of growth and/or number of hypomorphic ipl1 mutant yeast cells exposed to the test compound may be compared to the amount of growth and/or number of a culture of untreated hypomorphic ipl1 mutant yeast cells. For example, such hypomorphic ipl1 mutant yeast cells may include ipl1-321 or ipl1-2. According to these embodiments, hypomorphic ipl1 mutant yeast cells may be initially present in their respective mediums at approximately equal amounts or concentrations. After a predetermined period of time has elapsed, the amount of growth and/or number of cells within each culture may be determined. According to embodiments of present screening methods, yeast cells may be grown at a permissive temperature, such as any temperature in a range of about 23° C. to about 26° C., or at any temperature where the rate of growth of hypomorphic ipl1 mutant yeast cells is generally unaffected in the absence of a test compound. For example, such permissive temperature may be about 25° C. The predetermined period of time to allow for growth of yeast cells may vary depending on conditions and the volume of medium used. For example, a predetermined period of time of about 24 hours may be used.

Alternatively, according to some embodiments, yeast cells may be grown at a partially restrictive temperature, such as any temperature in a range of about 26° C. to about 35° C., or alternatively in a range of about 26° C. to about 29° C., or any temperature above the permissive temperature range where the rate of growth of hypomorphic ipl1 mutant yeast cells in the absence of a test compound is the same or approximately the same as wild-type yeast cells or the same or approximately the same as hypomorphic ipl1 mutant yeast cells grown at a permissive temperature, but where the hypomorphic ipl1 mutant yeast cells may have a greater sensitivity to test compounds that effectively inhibit Ipl1 expression and/or activity.

Yeast cells may be either grown in a liquid sample, solution, suspension, etc., or on a solid medium. According to some embodiments where a liquid medium is used, saturated or diluted concentrations of wild-type yeast cells or hypomorphic ipl1 mutant yeast cells or strains may be introduced into a solution, suspension, etc. containing adequate nutrients for growth. For example, where wild-type yeast cells or hypomorphic ipl1 mutant yeast cells are grown in a liquid medium, a YPD medium (about 1% yeast extract, about 2% peptone, and about 2% glucose) may be used. According to those embodiments where diluted concentrations of wild-type yeast cells or hypomorphic ipl1 mutant yeast cells, a 1/100 dilution, for example, may be used. According to embodiments of present methods, such medium may further contain a test compound that is to be tested or analyzed.

To measure an amount of growth, or a growth rate, of individual colonies or cultures of yeast cells in a liquid medium, or on a solid medium, with or without exposure to a test compound, different assays may potentially be used. Where a medium is a liquid sample, solution, suspension, etc., such as YPD, is used, growth of colonies or cultures of yeast cells in the liquid medium or culture may be determined by measuring the absorption of light by the liquid medium sample, solution, suspension, etc., using a spectrophotometer since the presence of yeast cells in the liquid medium will cause absorption of light at particular frequencies. For example, absorbance of light having a wavelength of about 600 nm (i.e., OD) may be measured to provide a measure of opacity and hence the number, concentration, and/or density of yeast cells in the liquid culture or medium.

Alternatively, yeast cultures may be grown on a solid medium or substrate, such as, for example, agar plates. Where yeast cells are grown on a solid medium or substrate, growth may be qualitatively measured and compared by visual inspection of plates to observe readily observable differences, or growth on solid substrates or media may be more exactly measured and compared, such as by measuring an area of growth or yeast cells, or by measuring a zone of inhibition of growth of yeast cells, in response to a test compound. Yeast cells may be introduced to a surface of a solid medium and a test compound may be added to such a medium to determine its effect on growth of such yeast cultures. A test compound could potentially be added before or after introduction of a population of yeast, and the test compound may be placed into or within a solid medium or substrate or placed on top of the solid medium or substrate. For example, according to some embodiments of the present screening methods, a piece of paper soaked in a solution containing a predetermined concentration of a test compound may be overlaid onto a solid medium having yeast cells of a particular genotype previously introduced onto such medium. The size of such piece of paper may correspond to only a small portion of the total area of such solid medium. The piece of paper containing a test compound may be left overlaid onto such solid medium for a sufficient period of time to affect the amount of growth of yeast cells or for the entire predetermined period of time for growth according to the particular embodiment, such as, for example, about 24 hours.

To measure an amount of growth or a growth rate of an individual culture of yeast cells grown on a solid medium after exposure to a test compound, different assays may potentially be used. For example, small pieces of material or paper (e.g. approximately 5 mm diameter cut circles) soaked in a solution containing a predetermined concentration of a test compound may be overlaid onto a predetermined position of a solid medium having a population or culture of yeast cells. Since the test compound is introduced to a particular location or position on the solid medium, it will be present at a highest concentration at such position of the solid medium (i.e., at, near, approaching, etc., the concentration of the test compound in the soaked piece of paper). The concentration of the test compound may also be present at radially decreasing concentrations at increasing distances from the position where the piece of material or paper is applied or overlaid as a result of diffusion. Thus, a gradient of concentrations of the test compound as a function of distance may be formed radially around the original point or position of application of the test compound. After a predetermined period of time for growth of the yeast cultures, growth inhibition may be measured on the solid medium as an area or zone of inhibition of growth within a lawn of yeast surrounding the original point or position of application of the test compound. Test compounds that are more potent at inhibiting growth of yeast cultures may produce a relatively larger area or zone of inhibition (i.e., it will inhibit growth at lower concentrations), whereas test compounds that are less potent at inhibiting growth of yeast cultures may produce a relatively smaller area or zone of inhibition (i.e., it will inhibit growth at only higher concentrations).

Therefore, the area or size of a zone of inhibition for a given test compound may provide a dynamic measurement of the effect and potency of such test compound on a particular yeast culture over a range of test compound concentrations. A zone of inhibition may be measured in terms of area, diameter, radius, circumference, etc. A zone of inhibition may be defined as an area, etc., of total inhibition of growth (i.e., no yeast cells are present) or an area, etc., of partial inhibition having a density of cells below a certain amount or threshold. According to some embodiments, a test compound may be determined to be a putative Aurora kinase inhibitor if the zone of inhibition for hypomorphic ipl1 mutant yeast cells is greater than the zone of inhibition for wild-type yeast cells.

When compared to initial values, growth data obtained according to embodiments of the present screening methods may provide a measure of growth for individual cultures or populations of yeast cells whether grown on or in solid or liquid media. According to some embodiments, for example, an amount of growth may be measured as a total change in the number, concentration, etc., of yeast cells with or without exposure to a test compound. For example, the total increase in the number, concentration, etc., of cells in a population or culture of yeast cells may be used to determine an amount of growth or a growth rate by such cells with or without exposure to a test compound. Alternatively, for example, growth may be measured at multiple time points to determine a growth rate by a population of yeast cells. Such growth rates or amounts of growth may be used to compare different cultures of yeast cells, such as wild-type and hypomorphic ipl1 mutant yeast cultures, in response to a particular test compound or a combination of test compounds to determine whether such compound(s) may comprise an inhibitor candidate or putative Aurora kinase inhibitor(s).

According to embodiments of present methods, a test compound may be determined to be a putative Aurora kinase inhibitor if the test compound causes complete or partial inhibition of growth of a population of hypomorphic ipl1 mutant yeast cells. If a test compound partially inhibits growth of a yeast culture, such a test compound may result in slow growth or a reduced number or density of yeast cells, whereas if a test compound completely inhibits growth of a yeast culture, such a test compound may result in complete or nearly complete absence of yeast cells. According to some embodiments, a test compound may be determined to be a candidate inhibitor, or a putative Aurora kinase inhibitor, if it results in either (i) slow growth and reduced numbers or density of yeast cells below a certain amount or threshold, or (ii) complete or nearly complete absence or lack of growth, of hypomorphic ipl1 mutant yeast cells in the presence of such test compound relative to wild-type yeast cells or untreated hypomorphic ipl1 mutant yeast cells after allowing growth in culture for a predetermined period of time.

According to embodiments of present screening methods, once quantitative measurements of amounts of growth for individual yeast cultures have been obtained, certain comparisons may be made to determine if a test compound may comprise a putative Aurora kinase inhibitor. For example, an amount or rate of growth of a culture of hypomorphic ipl1 mutant yeast cells, such as ipl1-321 or ipl1-2 mutant yeast cells, in response to treatment with a test compound may be compared to an amount or rate of growth of a culture of hypomorphic ipl1 mutant yeast cells, such as the same hypomorphic ipl1 mutant yeast cells, in the absence of treatment with such a test compound. This may be especially useful when a test compound(s), or a concentration of a test compound(s), is known or expected to not significantly affect the growth, division, and/or viability of a population of yeast cells under normal conditions. According to these embodiments, a test compound may be determined to be a putative Aurora kinase inhibitor if such a test compound results in a greater inhibition of an amount of growth of ipl1 mutant yeast cells treated with such test compound relative to untreated ipl1 mutant yeast cells.

According to some embodiments of present screening methods, an amount of growth of a culture of hypomorphic ipl1 mutant yeast cells, such as ipl1-321 or ipl1-2 mutant yeast cells, in response to treatment with a test compound, may be compared to an amount of growth of a culture of wild-type yeast cells in response to treatment with the test compound. According to these embodiments, a test compound may be determined to be a putative Aurora kinase inhibitor if such a test compound results in a greater inhibition of an amount of growth of ipl1 mutant yeast cells treated with such test compound relative to wild-type yeast cells treated with the same test compound, or alternatively, a test compound may be determined to be a putative Aurora kinase inhibitor if the amount of growth of the population of hypomorphic ipl1 mutant yeast cells is lower than the amount of growth of the population of wild-type yeast cells in response to the test compound.

According to some embodiments of present screening methods, once an amount of growth of a culture of wild-type yeast cells and an amount of growth of a culture of hypomorphic ipl1 mutant yeast cells are determined in the presence of a predetermined concentration of a given test compound, such test compound may be selected or determined to be a candidate or putative Aurora kinase inhibitor on the basis of certain criteria, such as by comparing the amounts of growth. According to some embodiments, a test compound may be determined to be a putative Aurora kinase inhibitor if there is a statistically significant difference, such as by t-test analysis, etc., between data sets in wild-type and hypomorphic ipl1 mutant yeast cells. For example, a test compound may be determined to be a putative Aurora kinase inhibitor if the amount of growth inhibition of hypomorphic ipl1 mutant yeast cells, such as ipl1-321 or ipl1-2 mutant yeast cells, is, for example, at least about 1.5, 2, 2.5, or 3 fold greater compared to wild-type yeast cells in the presence of a predetermined concentration of the test compound. The concentration of the test compound may be varied depending on a variety of factors, such as the desired stringency or sensitivity of the screen.

Alternatively, a test compound may be selected or determined to be a putative Aurora kinase inhibitor by comparison if the amount of growth of hypomorphic ipl1 mutant yeast cells, such as ipl1-321 or ipl1-2 mutant yeast cells, is, for example, at least about 60%, 50%, 40%, or 30% less or lower than the amount of growth of wild-type yeast cells in the presence of a predetermined concentration of the test compound. The concentration of the test compound may be varied depending on a variety of factors, such as the desired stringency or sensitivity of the screen.

According to some embodiments, a test compound may be determined to be a putative Aurora kinase inhibitor on the basis of selective inhibition of hypomorphic ipl1 mutant yeast cells relative to wild-type cells at a single predetermined concentration of the test compound or alternatively over a range of predetermined concentrations of the test compound.

According to some embodiments of present screening methods, a putative Aurora kinase inhibitor may also be identified or determined by comparing a growth differential between wild-type yeast cells and hypomorphic ipl1 mutant yeast cells by a test compound. The growth differential for wild-type yeast cells and hypomorphic ipl1 mutant yeast cells, such as ipl1-321 or ipl1-2 mutant yeast cells, may be defined as the difference in growth between treated and untreated populations of wild-type yeast cells and populations of hypomorphic ipl1 mutant yeast cells, respectively. According to these embodiments, a test compound may be determined to be a putative Aurora kinase inhibitor if the test compound causes a growth differential for hypomorphic ipl1 mutant yeast cells that is greater than a growth differential for wild-type yeast cells. According to some embodiments, to facilitate comparison of growth differential values, a growth differential may be expressed as an absolute value of the difference in the amounts of growth between treated and untreated populations of cells of the same type to ensure that only the magnitude of change in the amounts of growth are compared without regard to sign or order of subtraction.

Compounds that may be tested by embodiments of the present screening methods may comprise a class (or classes) of compounds from any known library of compounds, such as the Microbial Natural Product Database (MNPD). Alternatively, compounds tested by embodiments of the present screening methods may include newly synthesized and/or previously unidentified compounds. To increase the likelihood that compounds identified by embodiments of the screening methods of the present invention may represent bona fide Aurora kinase inhibitors, classes of compounds may be pre-screened on the basis of other criteria. For example, compounds subjected to embodiments of the present screening methods may be pre-selected on the basis of their known structure, such as according to computer programs that predict binding of compounds to a region of a target protein, such as an Aurora kinase or Ipl1, on the basis of conformational shape and positioning of critical residues or groups. According to some embodiments, compounds subjected to embodiments of the present screening methods may be pre-selected on the basis of their predicted ability to bind to an Aurora kinase enzyme active site, such as an ATP-binding site or pocket present within an Aurora kinase enzyme. An example of a structure-based virtual screen is described below. Alternatively, compounds may be pre-selected on the basis of an identical or analogous chemical structure to a known Aurora kinase inhibitor(s) or perhaps to compounds known to inhibit other kinase enzymes.

Each compound may be tested in replicate, and different concentrations of test compound may be tested and compared. Test compound concentrations of about 0.25, 0.5, 0.75, 1.0, 1.25, 2.5, 5, 7.5, 10, 15, 20, 40, 50, 60, 80, or 100 μg/ml may be used to measure an amount of growth for hypomorphic ipl1 mutant yeast cells and wild-type yeast cells in response to such test compound. Alternatively, concentrations of a test compound of from about 10⁻¹⁰ to about 10⁻⁴ M may be used. According to some embodiments, a test compound may be tested individually or in combination with other test compound(s). According to some embodiments, a compound may be tested over a range of concentrations to determine if there is a dose-responsive effect. According to some embodiments, an amount of growth for hypomorphic ipl1 mutant yeast cells and/or wild-type yeast cells may be measured at multiple time points following introduction of a test compound to provide a time-course plot for growth for each yeast culture. According to some embodiments, two or more growth mediums containing the same or different types of yeast cells with or without a test compound that are to be directly compared may contain an amount or concentration of a test compound that is about equal.

Due to the short doubling time of yeast cells and the ability to easily monitor growth of yeast cells in culture, embodiments of the screening methods of the present invention may be used to screen a large number of compounds over a short period of time. Various compounds may be tested simultaneously in different vials, tubes, dishes, multi-well plates, etc. Since test compounds may be selected or determined to be putative Aurora kinase inhibitors according to embodiments of present screening methods on the basis of their ability to preferentially or selectively inhibit the growth, division, viability etc., of hypomorphic ipl1 mutant yeast cells, compounds identified by embodiments of present screening methods may specifically target or inhibit Ipl1 expression or activity directly. Furthermore, given the high degree of homology between Ipl1 and Aurora kinases, such candidate compounds identified by embodiments of present screening methods may also show inhibition of Aurora kinases in higher organisms in many cases.

According to some embodiments of the present screening methods, test compounds may be tested individually or simultaneously in combination with other test compounds, and test compounds may be tested individually or simultaneously at multiple concentrations. Due to the advantages provided by using yeast cells to screen test compounds, embodiments of the present screening methods may easily accommodate high-throughput and efficient methods for screening large numbers of test compounds at perhaps multiple concentrations in a relatively short period of time. Indeed, embodiments of the present screening methods may accommodate the use of robots and other equipment to automate screening of numerous compounds in different vials, tubes, dishes, multi-well plates, such as 96 well plates, etc. Embodiments of the screening methods of the present invention may be carried out by computers under the direction of a software program(s) using cameras, spectrophotometers, or other known devices to measure or otherwise monitor growth rates of yeast cells, record values, and/or determine which compounds selectively affect growth of hypomorphic ipl1 mutant yeast cells. Such computer software may directly control and/or coordinate the operation of various components of such an apparatus.

Therefore, a high-throughput screening of test compounds may be carried out by embodiments of the present methods through computerized, automated, web-based, Internet-based, WAN-based, LAN-based, etc., methods to quickly and efficiently identify or determine compounds that selectively inhibit growth of hypomorphic ipl1 mutant yeast cells relative to wild-type yeast cells or untreated hypomorphic ipl1 mutant yeast cells. In addition, computer software may be used in combination with such automated, web-based, Internet-based, WAN-based, LAN-based, etc., performance of embodiments of the present screening methods to not only measure amounts of growth of various yeast cultures, but also to compare amounts of growth of various yeast cultures and determine if a test compound selectively inhibits growth of hypomorphic ipl1 mutant yeast cells relative to wild type yeast cells. For example, growth rates of various yeast cultures in response to such test compound(s) and/or range of predetermined concentrations of such test compound(s) may be measured via spectrophotometric analysis, such as by measuring OD₆₀₀, of yeast cultures in multi-well plates. Thus, embodiments of the present screening methods may use computerized, automated, web-based, Internet-based, WAN-based, LAN-based, etc., methods to quickly and efficiently determine which test compounds are putative Aurora kinase inhibitors and return or present to a user the identity or identities of test compound(s) that are putative Aurora kinase inhibitors.

Once a candidate or putative Aurora kinase inhibitor compound(s) is identified by embodiments of the present screening methods for their ability to inhibit growth of a culture or population of hypomorphic ipl1 mutant yeast cells, such compound(s) may be subsequently further characterized, tested, or confirmed by a variety of known methods, techniques, kits, etc., to determine if such putative Aurora kinase inhibitor compounds constitute bona fide Aurora kinase inhibitors in higher organisms. For example, such compounds may be further tested for their ability to inhibit Aurora kinase activity in vitro, to limit growth or cause cell cycle arrest of mammalian or human cells in culture, to cause apoptosis in treated cells, to inhibit phosphorylation of histone H3 in cells, etc. In addition, to determine if a putative Aurora kinase inhibitor identified by embodiments of the present screening methods is a bona fide inhibitor of the Ipl1 kinase enzyme itself, a number of known assays may be performed to determine if the presence of the putative Aurora kinase inhibitor inhibits the activity of Ipl1, such as inhibition of the ability of Ipl1 to phosphorylate known substrates (e.g., Dam1). See, e.g., Li, Y. et al., “The mitotic spindle is required for loading of the DASH complex onto the kinetochore,” Genes Dev 16(2):183-97 (2002), the entire content and disclosure of which is hereby incorporated by reference.

A wide variety of methods, techniques, assays, kits, etc., are available and known in the art that may be used to assay, measure, or determine expression, activity, and/or function of Aurora kinases in higher organisms when exposed to test compounds identified or determined to be putative Aurora kinase inhibitors by embodiments of the present screening methods, and some examples of such techniques, methods, assays, kits, etc., are described herein. For example, any known mammalian cell line(s) or cancer cell line(s), such as Hela, A549, or MCF-7 to name a few, may be used to assay for growth, division, viability, etc. of these cells in culture; Aurora kinase activity may be assayed by an Aurora-B kinase activity assay kit (e.g., Cell Signaling Technology; Danvers, Mass.); cell cycle arrest and/or apoptosis of cells may be assayed by FACS analysis; apoptosis may be assayed by fluorescence microscopy using a nuclear or DNA labeling dye, such as Hoechst 33342 or DAPI; histone H3 phosphorylation at Ser10 may be assayed by an anti-phospho H3 antibody; etc.

In addition, a test compound(s) determined to be a putative Aurora kinase inhibitor by embodiments of the present screening methods may be further examined in animal models to test their ability to counteract progression of cancerous cells or tumors. For example, putative Aurora kinase inhibitor compounds may be further tested or characterized for their ability to inhibit cancerous cells or tumors in whole animal models for disease, e.g. rats, mice, monkeys, etc., that develop cancer as a result of engineered mutations, transgenes, etc., or for their ability to inhibit cancerous cells or tissues being exogenously introduced into the body of an animal, such as by xenograft transplantation. Numerous examples of animal cancer models are known in the art. Putative Aurora kinase inhibitor compounds may be further selected on the basis of their ability to block the progression of disease in such animal models, such as by reducing the size or number of tumors or cancerous cells, through the use of detectable markers or karyotyping, or any other technique known in the art for assaying the state or progression of disease or cancer in such animal models. Alternatively, a putative Aurora kinase inhibitor compound(s) may be further selected according to clinical criteria on the basis of their ability to block the progression of a disease in humans. Progression against disease in humans may be monitored and determined according to the judgment of the attending physician for the individual, subject, or patient using known and available clinical and/or pathological techniques and/or diagnostic tools and may include, for example, many of the same types of observations used in monitoring progress against disease in animal models or human patients.

Alternatively, a putative Aurora kinase inhibitor compound(s) identified by embodiments of the present screening methods may be further selected on the basis of their strength or potency as inhibitors of Aurora kinases or on the basis of their kinetics of inhibition of Aurora kinases. For example, only compounds having a particular range of Kᵢ values or IC₅₀ values may be selected for further development. Such values may be determined in relation to varying levels of enzyme substrate. The exact preferred values or thresholds may vary depending on the therapeutic index or relative level of side effects associated with any given test compound or putative Aurora kinase inhibitor. For example, inhibitory or kinetic values for a given test compound may be determined relative to a normal substrate of the enzyme, such as ATP, at varying concentrations. For example, such concentrations of ATP may include a range including about 25, 50, 75, 100, and 200 μM. Furthermore, a putative Aurora kinase inhibitor(s) identified or determined by embodiments of the present screening methods may comprise any competitive, uncompetitive, non-competitive, or mixed inhibitor of an Aurora kinase enzyme.

However, it is to be understood that the above description merely gives examples of post-screening approaches providing for further selection or determination of Aurora kinase inhibitors. Indeed, any known in vivo, in vitro, or cell culture method, technique, kit, reagent, etc., may be used to assay or measure (i) cell cycle arrest, (ii) apoptosis, (iii) changes or inhibition in kinase activity (or more particularly changes or inhibition Aurora kinase activity), (iv) binding to Aurora kinases, etc., to validate or confirm if a test compound determined by embodiments of present the screening methods to be a putative Aurora kinase inhibitor is a true and effective inhibitor of an Aurora kinase enzyme from a higher organism.

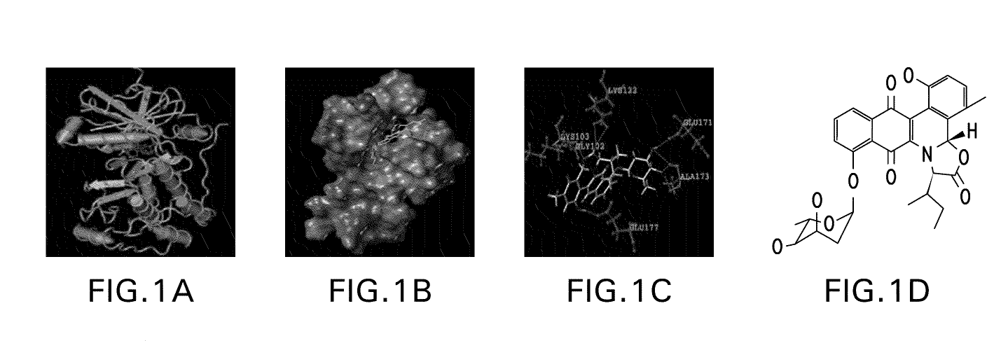

According to another broad aspect of the present invention, compositions may be provided comprising a test compound(s) identified or determined to be a putative Aurora kinase inhibitor(s) by embodiments of the present screening methods to selectively inhibit hypomorphic ipl1 mutant yeast cells relative to wild-type yeast cells. Such compositions may be formulated to inhibit an Aurora kinase enzyme in a higher organism. More particularly, embodiments of the compositions of the present invention may be formulated as pharmaceutical compositions comprising a therapeutically effective amount of a compound(s) identified or determined to be a putative Aurora kinase inhibitor(s) by embodiments of the present screening methods, or a salt, solvate, or hydrate thereof, in combination with a pharmaceutically acceptable carrier. As described herein, for example, some embodiments of the screening methods of the present invention have been used to identify Jadomycin B as an effective inhibitor of an Aurora kinase enzyme in human cells. Therefore, embodiments of the present compositions may comprise a therapeutically effective amount of Jadomycin B, or a salt, solvate, or hydrate thereof, in combination with a pharmaceutically acceptable carrier (the chemical structure of Jadomycin B is provided in FIG. 1D). Embodiments of the present compositions may further comprise a therapeutically effective amount of compound that is structurally related to Jadomycin B, or a salt, solvate, or hydrate thereof, in combination with a pharmaceutically acceptable carrier. Alternatively, embodiments of the compositions of the present invention may comprise a compound(s) unrelated to Jadomycin B identified by embodiments of the present screening methods.

Determination of a therapeutically effective amount of a compound may be carried out in a manner known to those skilled in the art. For example, therapeutically effective amount may comprise any appropriate dosage depending on the exigencies of a given situation. To determine an amount or dosage that is appropriate for administration to an individual, subject, or patient, treatment dosages may be titrated to optimize safety and effectiveness. Lower than expected dosages may be administered first to an individual, subject, and/or patient, and these dosages may then be titrated upward until a therapeutically effective and safe concentration amount (or a potentially unsafe concentration or amount) is reached.

Appropriate dosage amounts may be determined or predicted from empirical evidence. Dosages or concentrations tested in vitro for embodiments of the compounds of the present invention may provide useful guidance in determining therapeutically effective and appropriate amounts for in vivo administration. For example, a therapeutically effective dose of a compound identified by embodiments of the present screening methods may be estimated initially from a cell culture assay, for example, by measuring growth of mammalian cells in response to such compound, or from kinetics or equilibrium values for inhibition of an Aurora kinase enzyme in vitro, such as IC₅₀, Kᵢ, or other values. Such values may be used, for example, to translate into appropriate amounts for use in animal testing or for clinical trials in humans. Determining an appropriate dosage for embodiments of a compound or composition of the present invention may be discerned from any and/or all information or data available from any assay or experiment performed.

Animal testing of predicted dosages may provide additional indication of proper dosages for other types of animals, including humans. For example, a dosage of a compound identified by present screening methods that results in a circulating concentration that roughly approximates concentrations shown to be effective according to cell culture and/or in vitro assays may be used initially to determine effectiveness and/or safety at such concentration or to determine or extrapolate useful dosages for other animals, such as humans. Toxicity and therapeutic efficacy of any such compound may be determined or predicted from any standard pharmaceutical procedures based on data from cell cultures or experimental animals. For example, an LD₅₀ value (i.e., dose lethal in 50% of the population) and an ED₅₀ value (the dose therapeutically effective in 50% of subjects according to a certain criteria) may be determined for a given animal test subject, and the ratio of LD₅₀/ED₅₀ may be expressed as a therapeutic index. Compounds that exhibit a high therapeutic index may indicate that higher concentrations of test compounds or putative Aurora kinase inhibitors are safe and non-toxic and/or that lower doses may be efficacious in an individual, subject, or patient. However, a lower therapeutic index might indicate that only lower (and perhaps ineffective) concentrations of a compound may be acceptable in terms of safety. Levels of test compounds in the plasma of an individual, subject, or patient may be measured or monitored by any known technique including, for example, high performance liquid chromatography. In most cases, an appropriate dosage amount will be a balance of factors including efficacy and safety. Furthermore, an appropriate dosage amount will vary depending on the particular composition and mode of administration.

According to embodiments of present compositions, the exact formulation, route of administration, and dosage of the present compounds or compositions may be chosen according to the judgment of an ordinarily skilled scientist, veterinarian, or physician in view of the characteristics and conditions of an individual, subject or patient. The factors considered in determining a dosage that is therapeutically effective and safe for an individual, subject, or patient in clinical settings will depend on many of the same factors described above for a therapeutically effective amount (i.e., physical characteristics, gender, medical history, etc.). For appropriate considerations for determining appropriate dosage amounts, formulations, and/or routes of administration, see, e.g. Goodman and Gilman's, The Pharmacological Basis of Therapeutics, (11th ed., McGraw-Hill Professional, 2005); Remington, The Science and Practice of Pharmacy, (University of the Sciences in Philadelphia, 21st ed., Lippincott Williams & Wilkins, 2005); and Griffin P. et al., The Textbook of Pharmaceutical Medicine, (Blackwell Publishing, Malden, Mass. 2006), the contents and disclosures of which are hereby incorporated by reference.

According to some embodiments of the present invention, suitable concentrations or amounts of compounds or compositions may be administered in combination with a pharmaceutically acceptable carrier and/or a delivery agent to an individual, subject, or patient for the treatment, inhibition, prevention, management, etc., of a proliferative disease, such as cancer. Specific dosages may vary according to numerous factors and may be initially determined on the basis of in vitro, cell culture, and/or animal in vivo studies. Dosage amounts for embodiments of the compounds or compositions of the present invention may be in the range of, for example, from about 0.1 to about 100 mg/kg of body mass per day, from about 0.5 to about 60 mg/kg of body mass per day, or from about 1.0 to about 40 mg/kg of body mass per day. According to some embodiments, the unit dosage may be in the range of from about 1.0 to about 500 mg, from about 1.0 to about 250 mg, or from about 5.0 to about 150 mg. However, the exact dosage will depend on a consideration of relevant factors, including the relative levels of therapeutic effectiveness and safety, the mode of administration, etc., available empirical data about the compound or composition according to the knowledge and expertise of one skilled in the relevant art, etc.

Embodiments of the compounds or compositions of the present invention may be administered either as a single dose or as part of a dosage regimen. A dosage regimen may be adjusted to provide an optimum therapeutic response. For example, several different doses may be administered daily or doses may be proportionally reduced as indicated by the exigencies of the therapeutic situation. By administering an embodiment of a compound or composition of the present invention as part of a dosage regimen, circulating concentrations may be allowed to reach a desired equilibrium concentration for a compound through a series of doses. For convenience, a predetermined total daily dosage may be divided and administered in portions during the day as required. The compounds may be administered according to a dosage regimen of from about 1 to about 5 times per day, for example, 1, 2 or 3 times a day.

The mode or route of administration for embodiments of the compositions and compounds of the present invention may be selected to maximize delivery to a desired target site in the body of an individual, subject, or patient. Pharmaceutical compositions may be administered in a number of ways, including any suitable enteral, parenteral, topical, or local mode or route, depending on whether local or systemic treatment is preferred and/or the specific area to be treated. Suitable enteral routes for administration may include oral, rectal, intestinal, and gastric. Suitable parenteral routes may include intravascular routes, such as intravenous (bolus and infusion), intrarterial, and intracardiac; mucosal routes, such as transmucosal (e.g. insufflation), sublingual, buccal, intranasal, pulmonary (e.g., inhalation), and vaginal; intracranial; intraocular; intrathecal; intraperitoneal; intramuscular; intradermal; subcutaneous; intramedullary; or intraosseus. Embodiments of the pharmaceutical compositions of the present invention may be further administered via topical or transdermal routes as well as by local injection at a desired site of action, including peri- and intra-tissue injections, such as at or near a site of a tumor or cancerous cells in the body of an individual, subject, or patient.

Embodiments of the pharmaceutical compositions of the present invention may be administered in a variety of unit dosage forms depending on the method of administration. For example, unit dosage forms suitable for oral administration include solid dosage forms, such as powders, granules, tablets, pills, capsules, suppositories, depots, and dragees, and liquid dosage forms, such as elixirs, syrups, suspensions, sprays, gels, lotions, creams, slurries, foams, jellies, ointments, salves, solutions, suspensions, tinctures, and/or emulsions. Because of their ease of administration, tablets and capsules may be used as an oral dosage unit form when solid pharmaceutical compositions are employed. Pharmaceutical compositions may further include time- or sustained-release formulations. For parenteral administration, pharmaceutical compositions of the present invention may be formulated as sterile solutions, emulsions, and suspensions. Pharmaceutical compositions for topical administration may further include patches (e.g. dermal patches), and pharmaceutical compositions for pulmonary administration may include aerosols.

Examples of pharmaceutically acceptable carriers and other suitable additives and adjuvants for pharmaceutical compositions that may be used in combination with embodiments of the compounds or compositions of the present invention for administration to an individual, subject, or patient include those known to those skilled in the pharmacological art. As used herein, the pharmaceutically acceptable carriers may be either liquid or solid and may include solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, fillers, diluents, disintegration agents, lubricants, sweetening agents, flavoring agents, dyes, glidants, wetting agents, etc., and combinations thereof. For a description of pharmaceutical compositions, carriers, etc. that may be used in formulating pharmaceutical compositions and compounds of the present invention, see, for example, Remington, Pharmaceutical Science, (17th ed., Mack Publishing Company, Easton, Pa., 1985); Goodman & Gillman, The Pharmacological Basis of Therapeutics, (11^{th} Edition, McGraw-Hill Professional, 2005); and Griffin P. et al., The Textbook of Pharmaceutical Medicine, (Blackwell Publishing, Malden, Mass., 2006), the contents and disclosures of which are hereby incorporated by reference. See also, for example, U.S. Pat. Nos. 7,390,808, 7,354,928, 7,348,325, 7,326,713, and 7,282,504 (the contents and disclosures of which are hereby incorporated by reference) for a description of suitable pharmaceutical compositions, carriers, etc., that may be used with pharmaceutical compositions and compounds of the present invention.

Except insofar as any conventional pharmaceutical carrier is incompatible with embodiments of the compounds or compositions of the present invention, their potential use in pharmaceutical compositions of the present invention is contemplated. Embodiments of the pharmaceutical compositions and formulations of the present invention may utilize different types of carriers depending on whether they are to be administered in solid, liquid or aerosol form and whether they need to be sterile for certain routes of administration, such as local or systemic injection or infusion.

Various delivery systems and reagents known in the art are also contemplated for use as carriers in combination with compounds identified by embodiments of the present screening methods to be a putative Aurora kinase inhibitor. Where appropriate, such delivery systems or reagents may include, for example, liposomes, microparticles or nanoparticles, microcapsules, emulsions, polymers, etc., or any combination thereof. Liposomes may be coated with opsonization-inhibiting moieties or molecules (e.g., PEG) to avoid detection by the immune system and may be specifically formulated and/or associated with other molecules, antibodies, or conjugates to improve delivery, intake, and/or specificity into specific tissues or cells. See, e.g., Szoka et al., “Comparative properties and methods of preparation of lipid vesicles (liposomes),” Ann. Rev. Biophys. Bioeng., 9:467 (1980); Immordino, M. L., “Stealth liposomes: review of the basic science, rationale, and clinical applications, existing and potential,” Int. J Nanomedicine 1(3):297-315 (2006); Samad, A., “Liposomal Drug Delivery Systems: An Update Review,” Current Drug Delivery 4(4): 297-305 (2007); and Gregoriadis, G., Liposome Technology (Three-Volume Set), (3^{rd} Ed., Informa Healthcare, 2006), the contents and disclosures of which are hereby incorporated by reference in their entirety. See also, e.g., U.S. Pat. Nos. 4,501,728, 4,837,028, and 5,019,369, the contents and disclosures of which are hereby incorporated by reference. According to some embodiments, compositions of the present invention may include compositions comprising Jadomycin B in combination with an acceptable and/or suitable delivery reagent.

According to yet another broad aspect of the present invention, methods are provided for treating, inhibiting, preventing, managing, etc., a proliferative disease or condition, such as cancer, in an individual, subject, or patient by administering compositions comprising a test compound(s) identified or determined according to embodiments of the present screening methods to be a putative Aurora kinase inhibitor to such individual. For example, such putative Aurora kinase inhibitor may be Jadomycin B, or a salt, solvate, or hydrate thereof, or derivatives of Jadomycin B or other compounds having structural similarities to Jadomycin B, or a salt, solvate, or hydrate thereof. According to some embodiments, compositions comprising a putative Aurora kinase inhibitor identified by embodiments of present screening methods may be administered to an individual, subject, or patient experiencing a proliferative disease, such as cancer. For example, compositions comprising a putative Aurora kinase inhibitor identified by embodiments of the screening methods may be administered to an individual, subject, or patient having abnormally growing or dividing cells. Therefore, according to some embodiments, an individual, subject, or patient may be identified as having or experiencing a proliferative disease, such as cancer, or having abnormally growing or dividing cells, and a composition comprising a putative Aurora kinase inhibitor may then be administered to such individual.

According to some embodiments, compositions comprising a putative Aurora kinase inhibitor may be administered to an individual, subject, or patient in need of modulation of Aurora kinase activity in certain diseased cell(s) within the body of the individual. For example, an individual, subject, or patient in need of modulation of Aurora kinase activity may be an individual having cells within their body that have an abnormal expression and/or activity of an Aurora kinase enzyme. An individual, subject, or patient in need of modulation of Aurora kinase activity may also be an individual experiencing or having a particular condition or disease that may be known to be responsive to treatment with an Aurora kinase inhibitor or a particular condition or disease that may be known to be associated with abnormal expression and/or activity of an Aurora kinase enzyme. Such abnormal expression and/or activity of an Aurora kinase enzyme may be determined according to any known technique, such as by genotyping, sequencing, measuring activity in vitro, antibody detection, etc. For example, an individual, subject, or patient in need of modulation of Aurora kinase activity may be an individual experiencing a particular condition or disease that is known to be associated with one or more mutations in an Aurora kinase gene, such as, e.g., breast cancer, colon cancer, pancreatic cancer, ovarian cancer, gastric cancer, etc., which have already been shown to correlate with mutations in an Aurora kinase gene. An individual, subject, or patient in need of modulation of Aurora kinase activity may also be an individual having abnormally dividing or growing cells, such as cancerous cells, which may be known or expected to respond favorably to inhibition of an Aurora kinase enzyme. Therefore, according to some embodiments, an individual, subject, or patient may be identified as being in need of modulation of Aurora kinase activity in certain diseased cell(s) within the body of the individual, and a composition comprising a putative Aurora kinase inhibitor may then be administered to such individual.

According to some embodiments of the present methods for treating, inhibiting, preventing, managing, etc., a proliferative disease, such as cancer, pharmaceutical compositions comprising a test compound identified or determined by embodiments of present screening methods to be a putative Aurora kinase inhibitor may be administered in combination with a pharmaceutically acceptable carrier to an individual, subject, or patient experiencing a proliferative disease, such as cancer. For example, such a putative Aurora kinase inhibitor may be Jadomycin B, or a salt, solvate, or hydrate thereof, or derivatives of Jadomycin B or other compounds having structural similarities to Jadomycin B, or a salt, solvate, or hydrate thereof. It is further envisioned that compositions comprising a test compound identified or determined to be a putative Aurora kinase inhibitor by embodiments of the present screening methods may be administered alone or in combination with other drugs or therapies and/or possibly in combination with other putative Aurora kinase inhibitors identified by embodiments of the present screening methods. Compositions administered according to embodiments of the present methods for treating, inhibiting, preventing, managing, etc. a proliferative disease, such as cancer, may have any formulation known or described herein and may be provided to an individual, subject, or patient via any route of administration known or described herein. It is further envisioned that a test compound(s) identified or determined to be a putative Aurora kinase inhibitor may be administered alone as a naked composition.

According to embodiments of the present methods for treating, inhibiting, preventing, managing, etc., a proliferative disease, such as cancer, after administration of a composition (or pharmaceutical composition) comprising a putative Aurora kinase inhibitor to an individual, subject, or patient, progress against such disease may be monitored or measured in terms of efficacy and safety in response to administration of the composition. For treatment, inhibition, management, prevention, etc., of cancer, progress against disease may be monitored, for example, by observing changes in the size or properties of tumors, in the number or properties of cancerous cells, or in the size or properties of secondary or metastatic tumor sites. Progress against disease may also be measured according to known genetic, molecular, or biochemical techniques. There are numerous research methods, reagents, and/or diagnostic kits and tools known and available in the art (e.g. molecular markers, genetic testing, labels, antibodies, karyotyping, etc.) for monitoring progress against various and specific types of disease, including cancer. In addition, progress against disease, such as cancer, may be monitored according to any known or established veterinary, medical, and/or pathology techniques (e.g., by observation of symptoms, etc.). Although such techniques, reagents, and/or diagnostic kits are numerous, it is envisioned that any known research method, reagent, and/or diagnostic procedure or kit, as well as any known or established veterinary, medical, research, and/or pathology technique, may be used in combination with present methods of the present invention to monitor progress against disease. Progress against a particular disease may be evaluated according to known and available methods and diagnostics chosen by a qualified physician, veterinarian, or scientist attending to the care or treatment of an individual, subject, or patient as the case may be.

Progress against a proliferative disease, such as cancer, may be monitored, for example, by taking biopsies or samples from an individual, subject, or patient and assaying directly or indirectly for the effects on cells and/or tissues of such biopsies or samples in response to treatment with a composition of the present invention. Such biopsies or samples may be taken, for example, from tissues or fluids containing diseased cells or tissues, such as cancerous cells or tissues. Assaying Aurora kinase activity (e.g., by measuring histone H3 phosphorylation, etc.), analyzing pathology of cells or tissues (e.g., by staining to observe cytological or histological features, mitotic indices, extent of apoptosis, etc.), may be used to monitor progress against type of disease, such as cancer. Indeed, any method for assaying the expression and/or activity of an Aurora kinase enzyme or any histological or pathological feature(s) of cell(s) and/or tissue(s) taken from an individual described herein or known elsewhere in the art may be used as a way for monitoring progress against disease by embodiments of compounds or compositions of the present invention.

### EXAMPLES

The following non-limiting examples of embodiments of the present invention describe the identification of a new Aurora kinase inhibitor, Jadomycin B, from microbial natural products in a manner consistent with methods of the present invention. In the following examples, one particular hypomorphic ipl1 mutant allele is used, namely ipl1-321, representing a temperature-sensitive mutant allele of ipl1 to demonstrate the utility of embodiments of present screening methods. On the basis of these observations, it is envisioned that any hypomorphic allele of ipl1 that that is able to grow under normal or permissive conditions may potentially be used in the embodiments of the present screening methods and used as a substitute for ipl1-321.

The available crystal structure of Aurora-B kinase is used for virtual database screening. See, e.g., Sessa, F. et al., “Mechanism of Aurora B activation by INCENP and inhibition by hesperadin,” Mol Cell 18:379-91 (2005), the entire content and disclosure of which is hereby incorporated by reference. From this virtual screen, 22 compounds are identified from nearly 15,000 microbial natural products as potential small-molecular inhibitors of human Aurora-B kinase. One compound in particular, Jadomycin B, inhibits growth of ipl1-321 temperature sensitive mutants more dramatically than wild-type yeast cells, raising the possibility that this compound is an Aurora kinase inhibitor. Subsequent biochemical and genetic analysis confirmed that Jadomycin B is an Aurora-B inhibitor that inhibits the Aurora kinase in a dose-dependent manner. In the examples below, it is shown that Jadomycin B blocks phosphorylation of histone H3 on Ser10 in vivo like other Aurora kinase inhibitors and induces apoptosis in tumor cells without obvious effects on cell cycle. These examples demonstrate, therefore, that embodiments of the screening methods of the present invention relying on ipl1-321 temperature-sensitive mutant yeast cells may be used to successfully and credibly identify novel compounds that effectively inhibit Aurora kinases in higher organisms. More particularly, the examples below demonstrate that Jadomycin B may provide a potential drug or therapy for the treatment of cancer by targeting mitotic cells that rely on Aurora kinase activity.

### Structure-Based Virtual Screening

To identify potential inhibitors of Aurora-B kinase, the crystal structure of Aurora-B solved at 1.8-Å resolution is retrieved from the Protein Data Bank (PDB ID code 2BFY). The compound database used in this virtual screening is Microbial Natural Product Database (MNPD) developed by Institute of Medicinal Biotechnology, Chinese Academy of Medical Sciences, which contains the structural information of ˜15,000 microbial natural products. In this study, the ATP-binding pocket of Aurora-B is the target of interest, and the active sites are defined as all atoms within a radius of 6.5 Å from Hesperadin co-crystallized in the ATP-binding pocket. A docking program FlexX encoded in SYBYL7.1 (Tripos Inc) that uses an incremental construction algorithm is applied to optimize the interaction between flexible ligands and the rigid binding sites. In order to obtain an optimal starting confirmation, all ligands are minimized using Tripos standard force field and Gasteiger-Hückel atomic partial charges with termination gradient assigned 0.42 J/(mol·nm) prior to docking. As for Aurora-B protein, all crystal water molecules are removed from the original structure, hydrogen is added using Biopolymer module in SYBYL, and standard AMBER atomic partial charges are assigned. During soft docking simulations, the free energy of binding is calculated mainly by the sum of hydrogen bonds and hydrophobic interactions. The sum of the lowest estimated free energy from various binding conformations of each ligand is calculated and ranked by CScore function in SYBYL with default parameters. 150 compounds with top docking score are selected as candidates. Among them, 22 compounds are obtained.

### Determination of the Activity of Selected Compounds Using Budding Yeast

Wild-type and ipl1-321 mutant yeast strains are used for this purpose. Saturated yeast cells are 1/100 diluted into YPD (1% yeast extract, 2% peptone, and 2% glucose) medium containing tested compound at final concentration of about 10 μg/ml. After incubation at about 25° C. for about 24 hrs, the growth of the yeast cultures in the presence of tested compounds is determined by measuring OD₆₀₀.

### In Vitro Aurora-B Kinase Activity Assays

To determine the inhibition of Aurora-B kinase activity by Jadomycin B, an Aurora-B kinase activity assay kit (Cell Signaling Technology) is used according to the manufacturer's instruction. Briefly, 100 ng of purified recombinant human Aurora-B kinase is added to a 100 μl reaction mixture containing 1× kinase buffer and 25 μM cold ATP in the presence of different concentrations of Jadomycin B (ranging from 10⁻⁴ M to 10⁻¹⁰ M). After incubation at room temperature for 15 min, biotinylated peptide substrate (Cell Signaling Technology) is added to each reaction mixture at a final concentration of 1.5 μM, and the mixtures are further incubated for 30 min. A parallel control experiment is performed under the same conditions without Jadomycin B. The reaction is stopped by addition of 50 μM EDTA (pH 8). The 25 μl reaction mixture is then transferred to a streptavidin-coated 96-well plate (PerkinElmer, Inc) and incubated at room temperature for 60 min. After washing three times with PBS/T, the phospho-PLK (Ser10) antibody (Cell Signaling Technology) is added to the plate for further incubation at 37° C. for 120 min. After washing, FITC-labeled secondary antibody (Santa Cruz) is added. After incubation at room temperature for 30 min, the plate is finally washed five times, and the fluorescence signal is determined with BMG Polarstar Galaxy (Germany) at 535 nm. The inhibition ratio by Jadomycin B at each concentration is calculated according to the following equation: % Inhibition=100×(1−counts_{treated}/counts_{control}). The inhibition curve is then fitted by Originpro7.5 program and the IC₅₀ value of Jadomycin B is determined.

To determine the mode of inhibition of Aurora-B by Jadomycin B, the kinase activity is also examined in the presence of different concentrations of ATP (25, 50, 75, 100, and 200 μM). The inhibition ratio of Aurora-B kinase activity by Jadomycin B at various concentrations is calculated as described above and the inhibition curve is fitted. The IC₅₀ value of Jadomycin B at each ATP concentration is then determined and a linear fit is made with all five IC₅₀ values. The Kᵢ is determined from the intercept of the plot of [ATP] versus IC₅₀ values (IC₅₀=Kᵢ/Kₘ[ATP]+Kᵢ).

### Cell Growth Assay

A549, Hela and MCF-7 cell lines are cultured in 96-well tissue culture plates at a cell density of 5,000 cells per well in RPMI-I640 or MEM containing 10% fetus bovine serum and 2 mM L-glutamine. After attachment overnight, the medium is replaced and cells are incubated with increasing concentrations of Jadomycin B or its two derivatives, Jadomycin S and T (ranging from 10⁻⁴ M to 10⁻⁸ M). After treatment for 48 hours, 3-(4,5-dimethylthiazol-2-yl)-2,5-diphenyltetrazolium bromide (MTT) assays are carried out in triplication. The concentration-viability curves are fitted and IC₅₀ values are determined. Cells are also plated at a cell density of 10,000 cells per well in six-well tissue culture plates. After attachment overnight, cells are treated with 0, 5, or 10 μg/ml Jadomycin B for 96 hours. Cells are detached every 24 hr by trypsinization to count the cell number. All experiments are repeated three times.

### Fluorescence-Activated Cell Sorting (FACS)

A549 cells in exponential growth phase are treated with 0 or 5 μg/ml Jadomycin B for up to 48 hours. Cells collected at the indicated times are fixed and stained with Propidium Iodide (PI). FACS analysis is performed to determine the percentage of apoptotic cells and cell cycle distribution by using the EPICS XL flow cytometer and System II software.

### Detection of Chromatin Condensation and Apoptotic Body

Hoechst 33342 is a fluorescence dye that can be embedded in DNA double helix and emit blue fluorescence when excited with UV light. Treated and untreated A549 cell samples with 5 μg/ml Jadomycin B for 48 hours are stained with 100 ng/ml Hoechst 33342 for 10 minutes in dark. Stained cells are visualized with a Nikon ECLIPSS TE2000-U fluorescence microscope (Japan) at 100× and 400× magnification. Apoptotic cells show brightly stained nuclei because of the chromosome condensation and the appeared apoptotic bodies.

### Western Blotting

A549 cells in the exponential growth phase are treated with 0, 1.25, 2.5, 5, 7.5, or 10 μg/ml Jadomycin B for 24 hours, while Hela and HepG2 cells are treated with 0 or 10 μg/ml of Jadomycin B. Cells are washed with cold PBS and whole-cell extracts are prepared with RIPA lysis buffer containing 60 μg/ml of PMSF. Equal amounts of protein (30 μg) are resolved with 15% SDS-PAGE. After transfer, the membranes are incubated overnight with primary antibodies (1:1000) against phosphorylated histone H3 (Ser10) (Cell Signaling Technology) and β-tubulin (Santa Cruz Biotechnology) at 4° C. The membranes are then incubated with HRP-conjugated goat anti-rabbit secondary antibody (Santa Cruz) at 1:200 followed by Chemiluminescence staining (Millipore).

Identification of Jadomycin B through Structure-Based Virtual Screening

The crystal structure of Aurora-B shows that Aurora-B is composed of two homotypical subunits. Each subunit has two protein kinase domains, the N-terminal lobe (N-lobe, residues 86-174) rich in β-strands and the C-terminal lobe (C-lobe, residues 175-347) that mainly consist of α-helix. The N-lobe offers the binding sites of nucleotides and kinase regulators, while the C-lobe contains the active center where substrates are docked and catalytic reactions occur. The ATP-binding pocket that is composed of several spatially close residues (Lys122, Lys103, Glu171, Ala173, Leu99, Val107 and Glu177) lies at the interface between the two lobes. FIG. 1A shows the crystal structure of Aurora-B with Hesperadin, a known Aurora-B competitive inhibitor docked in the ATP-binding pocket. The indolinone moiety of Hesperadin occupies the catalytic cleft with the oxygen and nitrogen atoms of this moiety hydrogen bonded to Glu171 and Ala173. See, e.g., Sessa, F. et al., “Mechanism of Aurora B activation by INCENP and inhibition by hesperadin,” Mol Cell 18:379-91 (2005). Also, the sulfur and oxygen atoms of the sulfonamide moiety are hydrogen bonded to Lys122 and Lys103, occupying the position where the α-phosphate of ATP should be. The central phenyl ring of Hesperadin occupies the entry site to the catalytic cleft by van der Waals contact with other residues.

The protein structure is extracted from the 2bfy.pdb file and structure-based virtual screening is performed. 150 compounds with top docking score are selected as candidates. Among them, 22 are obtained, including Jadomycin B, a natural product (MW 563) isolated from Streptomyces venezuelae (see FIG. 1D). The docking data show that Jadomycin B could fit into the catalytic cleft of Aurora-B kinase and bind strongly to the residues surrounding the cleft (see FIG. 1B). Elaborate docking indicates that Jadomycin B occupies the ATP-binding pocket of the active center of Aurora-B with multiple interactions with the residues around the pocket: the two hydroxyls of L-digitoxose are hydrogen bonded to Glu171 and Ala173, while the two oxygen atoms of the oxazolone ring are hydrogen bonded to Lys122 and Lys103, respectively. At the same time the hydroxyl of the solvent-exposed angular phenyl ring in Jadomycin B binds to Glu177 with a hydrogen bond, occupying the entry site to the catalytic cleft in Aurora-B (see FIG. 1C).

### The Growth of Yeast Cells is Inhibited by Jadomycin B

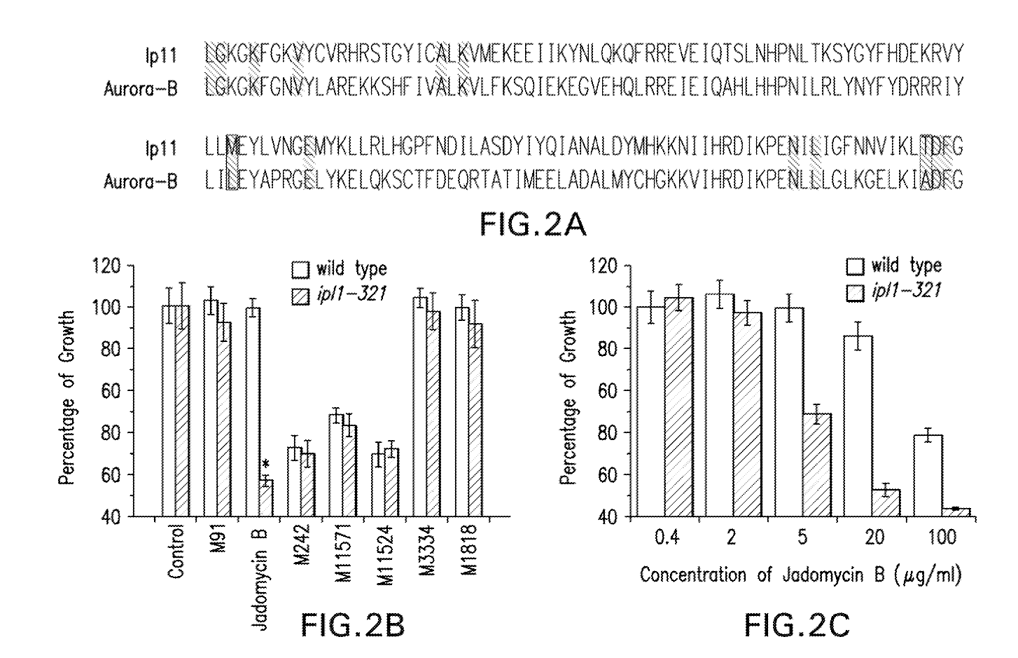

Aurora-B is a conserved protein kinase and its homologue in budding yeast is Ipl1. See, e.g. Kimura M et al. “Cell cycle-dependent expression and spindle pole localization of a novel human protein kinase, Aik, related to Aurora of Drosophila and yeast Ipl1” J Biol Chem 272:13766-71 (1997). It is reasoned that the ATP-binding pocket should be conserved well between these two kinases. As the crystal structure of Ipl1 is not available, the amino acid residues around the ATP binding site of Ipl1 kinase is compared with that of human Aurora-B and it is found that most of the residues are identical except two residues that play minor roles in ATP binding (see FIG. 2A). On the basis of this similarity, it is speculated that yeast Ipl1 kinase should also be sensitive to the identified Aurora-B inhibitors from the virtual screen.

Because Ipl1 kinase is essential for chromosome segregation, its inhibition will stop cell growth. ipl1-321 is a temperature sensitive mutant that grows well at 25° C. but fails to grow at the restrictive temperature (37° C.). See, e.g. Biggins, S. et al., “The budding yeast protein kinase Ipl1/Aurora allows the absence of tension to activate the spindle checkpoint,” Genes Dev 15:3118-29 (2001). Published data indicate that the Ipl1 kinase activity is compromised even when incubated at the permissive temperature 25° C. See, e.g. Kotwaliwale, C. V. et al., “A pathway containing the Ipl1/Aurora protein kinase and the spindle midzone protein Ase1 regulates yeast spindle assembly,” Dev Cell 13:433-45 (2007), the entire contents and disclosure of which is hereby incorporated by reference. It is reasoned that ipl1-321 mutant cells should exhibit more dramatic sensitivity to Aurora-B inhibitors. Therefore, the growth of wild-type and ipl1-321 cells is examined in the presence of 10 μg/ml of the 22 compounds, including Jadomycin B. After 24 hr incubation, the presence of 10 μg/ml of Jadomycin B inhibits the growth of ipl1-321 cells almost completely compared with the untreated control, while the growth of wild-type cells is uninhibited. Other compounds do not show any toxicity to yeast cells or exhibited similar toxicity to wild-type and ipl1-321 mutant cells (see FIG. 2B). The effect of two Jadomycin derivatives, Jadomycin S and Jadomycin T, on the growth of ipl1-321 mutant cells is also examined, but neither shows inhibitory effect at 10 μg/ml (data not shown).

To further determine whether the toxicity of Jadomycin B is due to its inhibition of Ipl1 kinase, the growth inhibition on wild-type and ipl1-321 mutant cells by Jadomycin B at different concentrations is compared. At 5 μg/ml, Jadomycin B inhibits 50% of the growth of ipl1-321 mutant cells, but there is little growth inhibition for wild-type cells. In the presence of 100 μg/ml of Jadomycin B, the growth of wild-type cells is inhibited by 60% (see FIG. 2C). The differential sensitivity of wild-type and ipl1-321 cells indicates that Jadomycin B could be an unidentified Aurora-B inhibitor since ipl1-321 cells are more sensitive to Jadomycin B.

### Jadomycin B Inhibits the Kinase Activity of Purified Recombinant Aurora-B

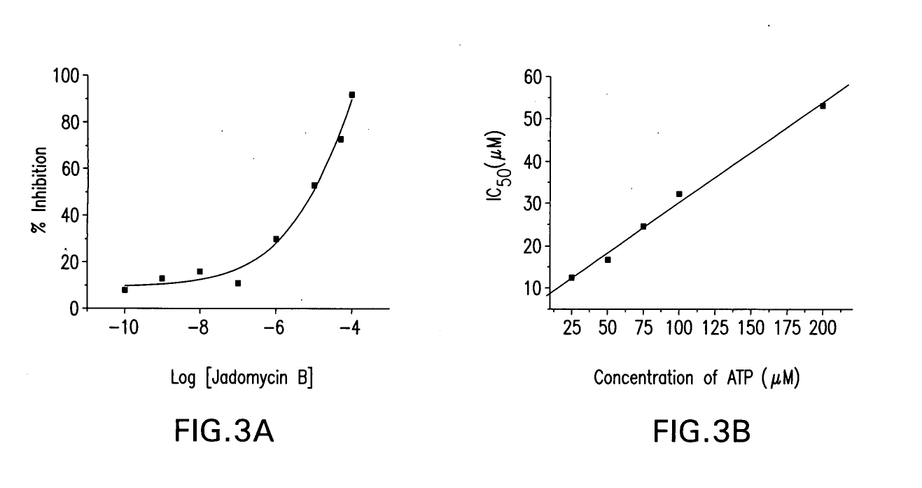

As the budding yeast Ipl1 kinase is the homologue of mammalian Aurora-B kinase, the result from yeast cells suggests that Jadomycin B might inhibit the kinase activity of mammalian Aurora-B. To test this possibility, the inhibition of purified human Aurora-B kinase by Jadomycin B using ELISA (Enzyme-linked ImmunoSorbent Assay) is examined. The kinase activity of Aurora-B, as indicated by fluorescence counts, is inhibited by micromolar concentration of Jadomycin B in a dose-dependent fashion, with an IC₅₀ value of 10.5 μM (see fitted inhibition curve in FIG. 3A). As controls, the inhibitory effect of Jadomycin S and Jadomycin T on Aurora-B under the same condition is also tested, but no inhibitory activity is detected (data not shown).

The mode of Jadomycin B-dependent inhibition of Aurora-B kinase is determined next. It is speculated that Jadomycin B inhibits Aurora-B by preventing the access of ATP to the kinase domain based on the structure analysis. Therefore, the inhibition of the kinase activity by Jadomycin B is analyzed in the presence of ATP at different concentrations (25, 50, 75, 100, or 200 μmol/L). The IC₅₀ values with a given concentration of ATP is determined by fitting the inhibition curve and they are 12.46 μM (25 μM ATP), 16.60 μM (50 μM ATP), 24.50 μM (75 μM ATP), 32.16 μM (100 μM ATP) and 53.09 μM (200 μM ATP) (see linear fit in FIG. 3B). The titration experiments using 5 different concentrations of ATP reveal a competitive inhibition of Aurora-B kinase by Jadomycin B with respect to ATP, and the Kᵢ of Aurora-B by Jadomycin B is 6.8 μM.

### Jadomycin B Inhibits the Proliferation of Cancer Cells

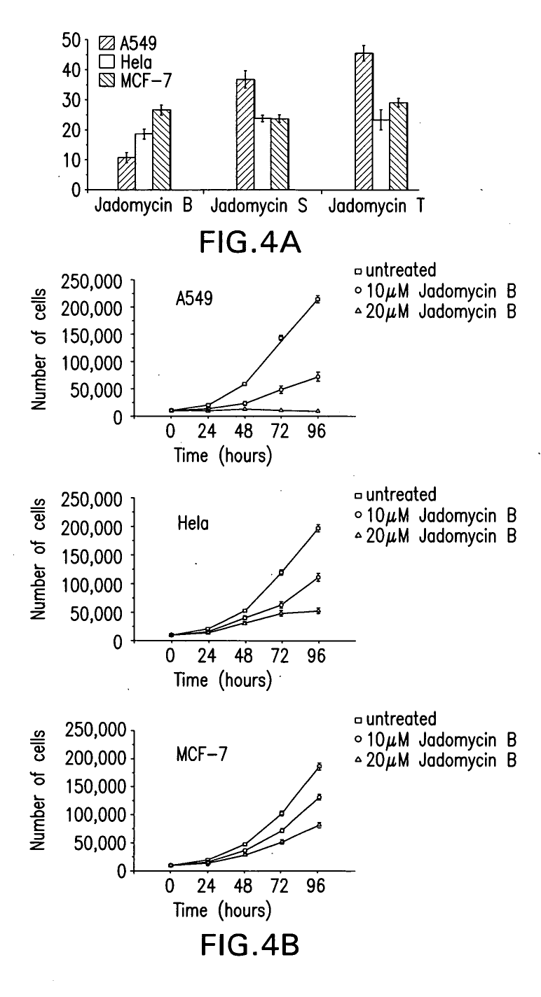

It has been reported that Jadomycin B inhibits the proliferation of IM-9, IM9/Bcl-2, HepG2 and H460 cells. See, e.g. Zheng, I. T. et al., “Cytotoxic activities of new jadomycin derivatives,” J Antibiot 58:405-8 (2005), the entire content and disclosure of which is incorporated by reference. The effects of Jadomycin B on the growth of other three cancer cell lines, A549, Hela, and MCF-7 is examined. The growth curves indicate that all these cell lines are sensitive to Jadomycin B and the growth inhibition is in a dose-dependent manner (see FIG. 4B). The IC₅₀ values of Jadomycin B and its two derivatives determined by MTT assays against these cancer cell lines are shown in FIG. 4A. Though A549 cells are most sensitive to Jadomycin B, the inhibitory potency does not differ significantly among these cell lines. Although Jadomycin S and T also inhibited growth of cancer cell lines, this is likely due to inhibition of targets other than Aurora B since Jadomycin S and T are shown to neither inhibit ipl1-321 cells or inhibit Aurora B.

Jadomycin B Induces Apoptosis without Blocking Cell Cycle

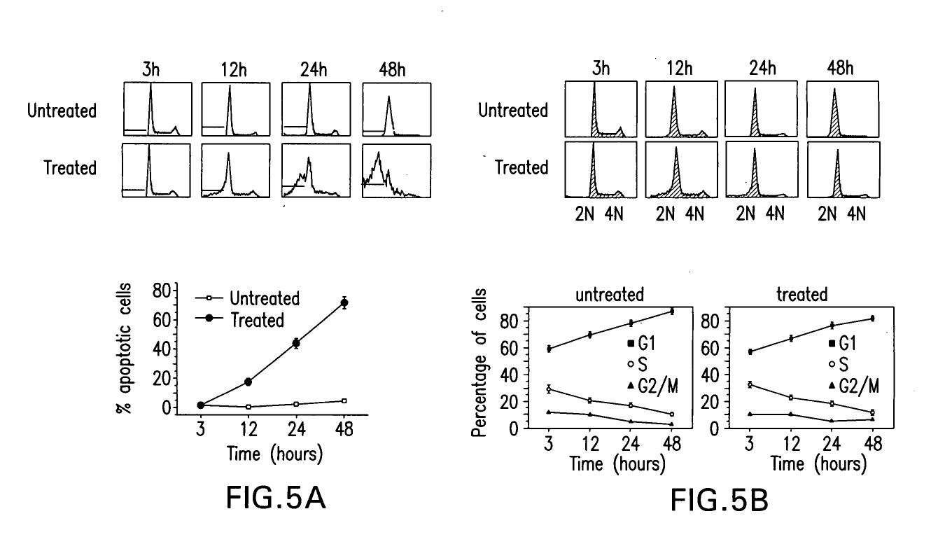

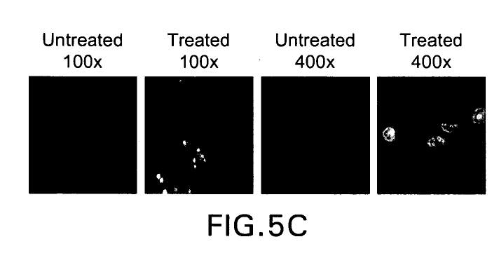

FACS analysis is used to examine the mechanism of cell growth inhibition by Jadomycin B. Jadomycin B is added to A549 cell cultures in the exponential growth phase to a final concentration of 5 μg/ml. Treated and untreated cell samples are taken at 3, 12, 24, and 48 hr and fixed for FACS analysis. Although treatment with 5 μg/ml of Jadomycin B induces an increase of cells in S phase by 2%, the difference between treated and untreated cells is so negligible that no obvious accumulation of cells in a specific cell cycle stage is observed, indicating that Jadomycin B does not block cell cycle at this concentration (FIG. 5B). Instead, an increased cell population with sub-G1 content of DNA is found (see FIG. 5A), suggesting that apoptosis occurs. After 3 hr incubation, there is almost no difference in the percentage of apoptotic cells between the treated and untreated cells. After longer exposure to Jadomycin B, however, the portion of apoptotic cells in treated samples increased to 17.8%, 44% and 71.7% at 12, 24, and 48 hr, respectively, while those of untreated sample remained at less than 5% (FIG. 5A). Hoechst 33342 staining confirms that more and more cells underwent apoptosis after long time exposure to Jadomycin B, as indicated by the appearance of brightly stained compressed chromosomes (FIG. 5C). Meanwhile, the apoptotic body could be detected at a 400× magnification under a fluorescence microscope (see FIG. 5C). The nuclei of apoptotic cells shrank, became round, and stained brightly with nuclear dyes. Therefore, 5 μg/ml of Jadomycin B induces apoptosis in A459 cells without cell cycle disturbance.

### Jadomycin B Inhibits Histone H3 Phosphorylation

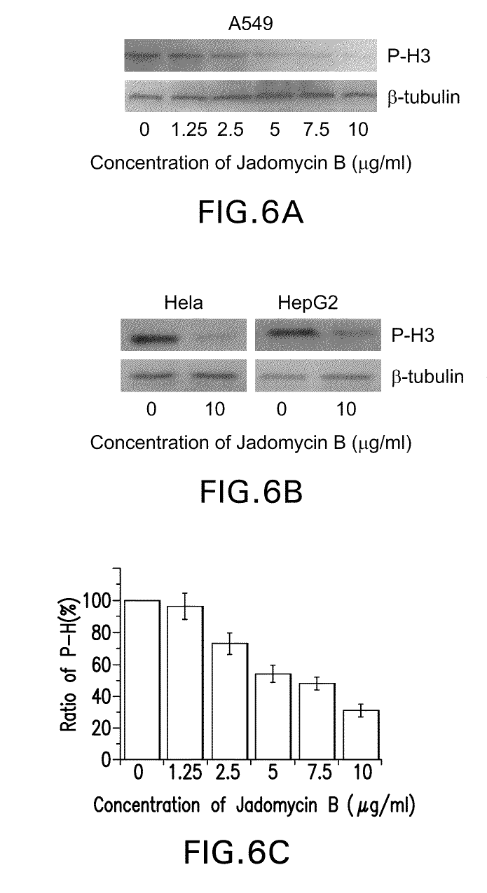

It has been demonstrated that Aurora-B kinase phosphorylates histone H3 on serine 10. It is reasoned that this phosphorylation should be impaired in the presence of Jadomycin B if it inhibits the activity of Aurora-B kinase. To test this idea, the phosphorylation of histone H3 in A549 cells after 24 hr treatment with Jadomycin B at different concentrations is examined. In untreated cells, a clear phospho-H3 band is observed after western blot analysis with anti-phospho-H3 antibody. In contrast, A549 cells do not express any detectable phosphorylated histone H3 after treatment with 10 μg/ml Jadomycin B (see FIG. 6A). A significant decrease in the levels of phosphorylated histone H3 upon treatment with Jadomycin B is also observed in Hela and HepG2 cells (see FIG. 6B). Further quantitative analysis demonstrates that Jadomycin B inhibits histone H3 phosphorylation in a dose-dependent manner. Treatment with 2 or 10 μg/ml Jadomycin B reduces H3 phosphorylation by 28% and 73%, respectively (FIG. 6C). Collectively, these data demonstrate that Jadomycin B inhibits the kinase activity of Aurora-B.

Further description of examples and/or embodiments of the present invention may be provided in Fu et al., “Jadomycin B, an Aurora kinase inhibitor discovered through virtual screening,” Mol Cancer Ther 7(8):2386-2393 (2008), the entire content and disclosure of which is hereby incorporated by reference.

While the present invention has been disclosed with references to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method comprising the following steps:
(a) measuring an amount of growth over a predetermined period of time of a population of wild-type yeast cells in or on a first medium containing a test compound and an amount of growth over the predetermined period of time of a population of hypomorphic ipl1 mutant yeast cells in or on a second medium containing the test compound; and
(b) comparing the amount of growth of the population of hypomorphic ipl1 mutant yeast cells to the amount of growth of the population of wild-type yeast cells to thereby determine whether the test compound is a putative Aurora kinase inhibitor.

2. The method of claim 1, wherein the population of hypomorphic ipl1 mutant yeast cells comprises ipl1-321 mutant yeast cells or ipl1-2 mutant yeast cells.

3. The method of claim 1, wherein prior to step (a), the test compound is introduced to the first medium and the second medium such that the concentration of the test compound in or on each of the first and second mediums is about equal.

4. The method of claim 1, wherein the population of wild-type yeast cells are introduced to the first medium and the population of hypomorphic ipl1 mutant yeast cells are introduced to the second medium in approximately equal amounts.

5. The method of claim 1, wherein the population of wild-type yeast cells in the first medium and the population of hypomorphic ipl1 mutant yeast cells in the second medium are each grown during the predetermined period of time at a permissive temperature.

6. The method of claim 5, wherein the permissive temperature is within a range of from about 23° C. to about 26° C.

7. The method of claim 1, wherein the population of wild-type yeast cells in the first medium and the population of hypomorphic ipl1 mutant yeast cells in the second medium are each grown during the predetermined period of time at a partially restrictive temperature.

8. The method of claim 7, wherein the partially restrictive temperature is within a range of from about 26° C. to about 35° C.

9. The method of claim 1, wherein prior to step (a), the test compound is introduced to the first medium before the population of wild-type yeast cells are introduced to the first medium, and the test compound is introduced to the second medium before the population of hypomorphic ipl1 mutant yeast cells are introduced to the second medium.

10. The method of claim 1, wherein prior to step (a), the population of wild-type yeast cells are introduced to the first medium before the test compound is introduced to the first medium, and the population of hypomorphic ipl1 mutant yeast cells are introduced to the second medium before the test compound is introduced to the second medium.

11. The method of claim 1, wherein the test compound is determined to be a putative Aurora kinase inhibitor if the test compound inhibits growth of the population of hypomorphic ipl1 mutant yeast cells to a greater extent than the test compound inhibits growth of the population of wild-type yeast cells.

12. The method of claim 11, wherein the test compound is determined to be a putative Aurora kinase inhibitor if the test compound inhibits growth of the population of hypomorphic ipl1 mutant yeast cells at least about 1.5 fold greater than the test compound inhibits growth of the population of wild-type yeast cells.

13. The method of claim 1, wherein the test compound is determined to be a putative Aurora kinase inhibitor if the amount of growth of the population of hypomorphic ipl1 mutant yeast cells is lower than the amount of growth of the population of wild-type yeast cells.

14. The method of claim 13, wherein the test compound is determined to be a putative Aurora kinase inhibitor if the amount of growth of the population of ipl1 mutant yeast cells is at least about 30% lower than the amount of growth of the population of wild-type yeast cells.

15. The method of claim 1, wherein the measuring step (a) comprises determining the amount of light absorbed by each of the first and second mediums.

16. The method of claim 15, wherein the light used in measuring step (a) has a wavelength of about 600 nm.

17. The method of claim 1, wherein the test compound is pre-selected on the basis of its chemical structure prior to step (a).

18. The method of claim 17, wherein the test compound is pre-selected on the basis of its resemblance to all or a portion of a chemical structure of a known kinase inhibitor.

19. The method of claim 17, wherein the test compound is pre-selected by a virtual screening software program.

20. The method of claim 17, wherein the test compound is pre-selected on the basis of a known chemical structure of an Aurora kinase enzyme.

21. The method of claim 20, wherein the known chemical structure of an Aurora kinase enzyme is a known chemical structure of an Aurora kinase enzyme active site.

22. The method of claim 20, wherein the known chemical structure of an Aurora kinase enzyme is a known chemical structure of an Aurora kinase enzyme ATP-binding pocket.

23. The method of claim 1, comprising the further step (c) of determining whether the putative Aurora kinase inhibitor inhibits growth of cells of a culture of mammalian cells treated with the putative Aurora kinase inhibitor relative to cells of an untreated culture of mammalian cells.

24. The method of claim 1, comprising the further step (c) of determining if the putative Aurora kinase inhibitor alters the cell cycle distribution of cells of a culture of mammalian cells treated with the putative Aurora kinase inhibitor relative to cells of an untreated culture of mammalian cells.

25. The method of claim 24, wherein the determining step (c) comprises performing FACS analysis on cells of the treated and untreated cultures of mammalian cells.

26. The method of claim 1, comprising the further step (c) of determining if cells of a culture of mammalian cells treated with the putative Aurora kinase inhibitor have increased apoptosis relative to cells of an untreated culture of mammalian cells.

27. The method of claim 26, wherein the determining step (c) comprises performing FACS analysis on cells of the treated and untreated cultures of mammalian cells.

28. The method of claim 26, wherein the determining step (c) comprises staining cells of the treated and untreated cultures of mammalian cells with a DNA labeling dye and counting the number of apoptotic cells by microscopy.

29. The method of claim 1, comprising the further step (c) of determining if the putative Aurora kinase inhibitor inhibits phosphorylation of histone H3 at Ser 10 in cells of a culture of mammalian cells treated with the putative Aurora kinase inhibitor relative to cells of an untreated culture of mammalian cells.

30. The method of claim 29, wherein the determining step (c) comprises performing Western blot analysis using an anti-phospho-histone H3 antibody on protein extracts derived from cells in a culture of mammalian cells.

31. The method of claim 1, comprising the further step (c) of determining if the putative Aurora kinase inhibitor inhibits the activity of an Aurora kinase enzyme in vitro.

32. The method of claim 1, comprising the further step (c) of determining if the putative Aurora kinase inhibitor inhibits phosphorylation of an Ipl1 substrate.

33. The method of claim 32, wherein the Ipl1 substrate is a yeast Dam1 protein.

34. The method of claim 1, wherein the amount of growth of the population of wild-type yeast cells and the amount of growth of the population of hypomorphic ipl1 mutant yeast cells measured in step (a) comprises a rate of growth during the predetermined period of time.

35. The method of claim 1, wherein the first and second mediums each comprise a solid medium.

36. The method of claim 35, wherein the test compound is introduced to the first medium and the second medium by overlaying a first piece of material soaked in a solution containing the test compound onto a portion of the first medium and overlaying a second piece of material soaked in a solution containing the test compound onto a portion of the second medium.

37. The method of claim 36, wherein the measuring step (a) comprises measuring the amount of growth of the population of wild-type yeast cells on the first medium by measuring a first zone of inhibition at or near the first piece of material and measuring the amount of growth of the population of hypomorphic ipl1 mutant yeast cells on the second medium by measuring a second zone of inhibition at or near the second piece of material.

38. The method of claim 37, wherein the test compound is determined to be a putative Aurora kinase inhibitor if the second zone of inhibition is greater than the first zone of inhibition.

39. A pharmaceutical composition comprising a therapeutically effective amount of a test compound in combination with a pharmaceutically acceptable carrier, wherein the test compound has been determined to be a putative Aurora kinase inhibitor according to the method of claim 1.

40. The pharmaceutical composition of claim 39, wherein the pharmaceutically acceptable carrier comprises a delivery reagent.

41. The pharmaceutical composition of claim 40, wherein the delivery reagent is selected from the group consisting of liposomes, microparticles, nanoparticles, microcapsules, emulsions, polymers, and combinations thereof.

42. The pharmaceutical composition of claim 39, wherein the putative Aurora kinase inhibitor comprises Jadomycin B, and wherein the pharmaceutical composition comprises an amount of Jadomycin B effective to inhibit an Aurora kinase enzyme.

43. A method, comprising the following steps:
(a) identifying an individual experiencing a form of cancer; and
(b) administering to the individual a composition comprising a therapeutically effective amount of a test compound, wherein the test compound has been determined to be a putative Aurora kinase inhibitor according to the method of claim 1.

44. The method of claim 43, wherein the composition is a pharmaceutical composition further comprising a pharmaceutically acceptable carrier.

45. The method of claim 43, wherein the test compound is Jadomycin B.

46. A method, comprising the following steps:
(a) identifying an individual in need of modulation of an Aurora kinase enzyme; and
(b) administering to the individual a composition comprising a therapeutically effective amount of a test compound, wherein the test compound has been determined to be a putative Aurora kinase inhibitor according to the method of claim 1.

47. The method of claim 46, wherein the composition is a pharmaceutical composition further comprising a pharmaceutically acceptable carrier.

48. The method of claim 46, wherein the test compound is Jadomycin B.

49. A method comprising the following steps:
(a) measuring over a predetermined period of time: (i) an amount of growth of a first population of wild-type yeast cells in or on a first medium containing a test compound; (ii) an amount of growth of a first population of hypomorphic ipl1 mutant yeast cells in or on a second medium containing the test compound; (iii) an amount of growth of a second population of wild-type yeast cells in or on a third medium lacking the test compound; and (iv) an amount of growth of a second population of hypomorphic ipl1 mutant yeast cells in or on a fourth medium lacking the test compound;
(b) calculating a growth differential for wild-type yeast cells by calculating the difference between the amount of growth of the first population of wild-type yeast cells and the amount of growth of the second population of wild-type yeast cells and a growth differential for hypomorphic ipl1 mutant yeast cells by calculating the difference between the amount of growth of the first population of hypomorphic ipl1 mutant yeast cells and the amount of growth of the second population of hypomorphic ipl1 mutant yeast cells; and
(c) comparing the growth differential for the population of hypomorphic ipl1 mutant yeast cells to the growth differential for the population of wild-type yeast cells to thereby determine whether the test compound is a putative Aurora kinase inhibitor.

50. The method of claim 49, wherein the test compound is determined to be a putative Aurora kinase inhibitor if the absolute value of the growth differential for the population of hypomorphic ipl1 mutant yeast cells is greater than the absolute value of the growth differential for the population of wild-type yeast cells.

51. The method of claim 49, wherein the population of hypomorphic ipl1 mutant yeast cells comprises ipl1-321 mutant yeast cells or ipl1-2 mutant yeast cells.

52. The method of claim 49, wherein the population of wild-type yeast cells in the first medium and the population of hypomorphic ipl1 mutant yeast cells in the second medium are grown during the predetermined period of time at a permissive temperature.

53. The method of claim 52, wherein the permissive temperature is within a range of about 23° C. to about 26° C.

54. The method of claim 49, wherein the population of wild-type yeast cells in the first medium and the population of hypomorphic ipl1 mutant yeast cells in the second medium are grown during the predetermined period of time at a partially restrictive temperature.

55. The method of claim 54, wherein the partially restrictive temperature is within a range of about 26° C. to about 35° C.

56. A method comprising the following steps:
(a) measuring an amount of growth over a predetermined period of time of a first population of hypomorphic ipl1 mutant yeast cells in or on a first medium containing a test compound and an amount of growth over the predetermined period of time of a second population of hypomorphic ipl1 mutant yeast cells in or on a second medium lacking the test compound; and
(b) comparing the amount of growth of the first population of hypomorphic ipl1 mutant yeast cells to the amount of growth of the second population of hypomorphic ipl1 mutant yeast cells to thereby determine whether the test compound is a putative Aurora kinase inhibitor.

57. The method of claim 56, wherein the test compound is determined to be a putative Aurora kinase inhibitor if the amount of growth of the first population of hypomorphic ipl1 mutant yeast cells is lower than the amount of growth of the second population of hypomorphic ipl1 mutant yeast cells.

58. The method of claim 56, wherein the population of hypomorphic ipl1 mutant yeast cells comprises ipl1-321 mutant yeast cells or ipl1-2 mutant yeast cells.
